(19) United States
(12) Patent Application Publication
(10) Pub. No.: US 2009/0104633 A1
(43) Pub. Date: Apr. 23, 2009

(54) Kit for Highly Sensitive Detection Assays

(76) Inventors: Steffen Bade, Klein Ronnau (DE); Niels Rockendorf, Bargfeld-Stegen (DE); Milan Franek, Brno (CZ); Hans-Heiner Gorris, Bedburg-Hau (DE); Andreas Frey, Gross Niendorf (DE)

Correspondence Address: NIXON & VANDERHYE, PC, 901 NORTH GLEBE ROAD, 11TH FLOOR, ARLINGTON, VA, 22203, US

(21) Appl. No.: 12/084,246

(22) PCT Filed: Oct. 31, 2006
(86) PCT No.: PCT/EP2006/010488

§ 371 Date: Apr. 28, 2008

(30) Foreign Application Priority Data
Oct. 31, 2005 (DE) 102005051976.8

(51) Int. Cl. G01N 33/53; G01N 33/543; C12P 19/34

(52) U.S. Cl. 435/7.92; 436/501; 436/518; 435/91.2

(57) ABSTRACT

The present invention relates to kits comprising 2,4-dichlorophenoxyacetate derivatives as well as antibodies that bind to these derivatives. Inter alia, the kits can be used to label biomolecules for analytical and diagnostic applications. Some of the compounds described here can be used to label biomolecules under physiological conditions and without having to apply in situ activation. Furthermore, the presence of spacers within the 2,4-dichlorophenoxyacetate derivatives improves their binding to antibodies.

The present invention relates to kits comprising novel 2,4-dichlorophenoxyacetate derivatives as well as antibodies that bind to these derivatives. Inter alia, the kits can be used to label biomolecules for analytical and diagnostic applications. Some of the compounds described herein can be used to label biomolecules under physiological conditions and without having to apply in situ activation. Furthermore, the presence of spacers within the 2,4-dichlorophenoxyacetate derivatives improves their binding to antibodies.

### BACKGROUND OF THE INVENTION

Technologies and applications of analytical and diagnostic methods are evolving rapidly. In the course of the majority of current procedures, the detection of substrate-molecules is of critical importance. Several methods are available to perform detection assays, e.g. the Biotin/Streptavidin-system or variants thereof (Symons R H, 1990, U.S. Pat. No. 4,898,951A, Compounds used as intermediates in the preparations of non-radioactive biological probes; Boehringer Mannheim, 1993, U.S. Pat. No. 5,219,764A, Hapten-biotin conjugates and their use) or Digoxigenin/Antibody-based approaches (Boehringer Mannheim, 1990, DE3836656A1, Neue Digoxigenin-Derivate und ihre Verwendung). Due to the great significance of substrate detection-assays at large and the frequent requirement for multiple labels in the course of procedures employed in current analytics and diagnostics it is therefore necessary to develop novel kits for detection assays.

In view of the state of the art the problem resides in providing novel kits for the detection of substrate molecules, e.g. amino acids, peptides, proteins, nucleotides, nucleic acids, saccharides or lipids.

This problem is solved by the present invention.

### DESCRIPTION OF THE INVENTION

The present invention is concerned with kits comprising at least one labelling compound represented by formula (I)

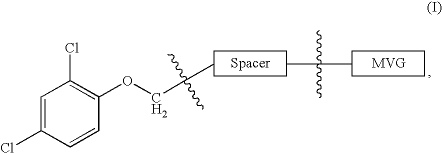

wherein the compound is stable in water and soluble, and wherein the “Spacer” comprises 1 to 25 identical or different protected or unprotected amino acids, nucleotides, saccharides, polyoles or residues selected from the following group:

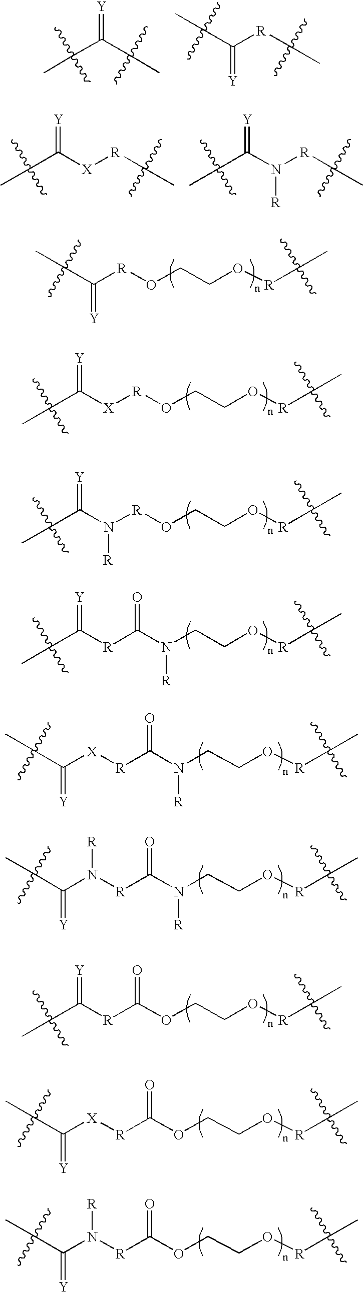

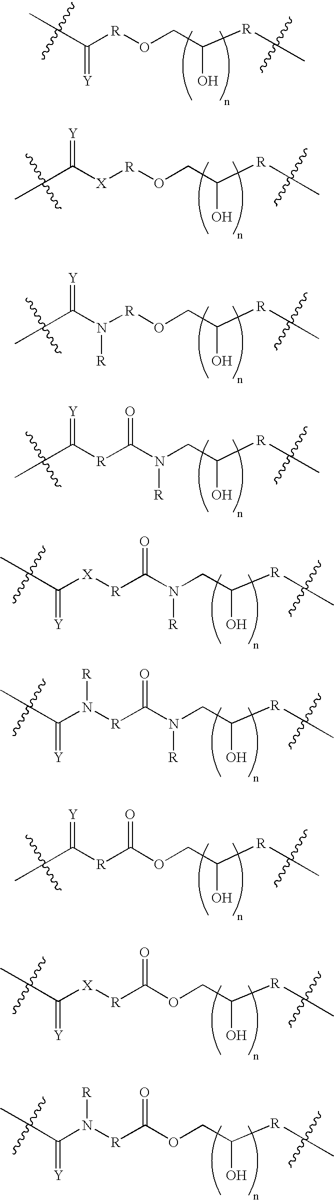

wherein X and Y, independently from each other, can be —O— or —S—, and n is an integer in the range of 1-15,
wherein the “label mediating group” (MVG) is selected from the following group:

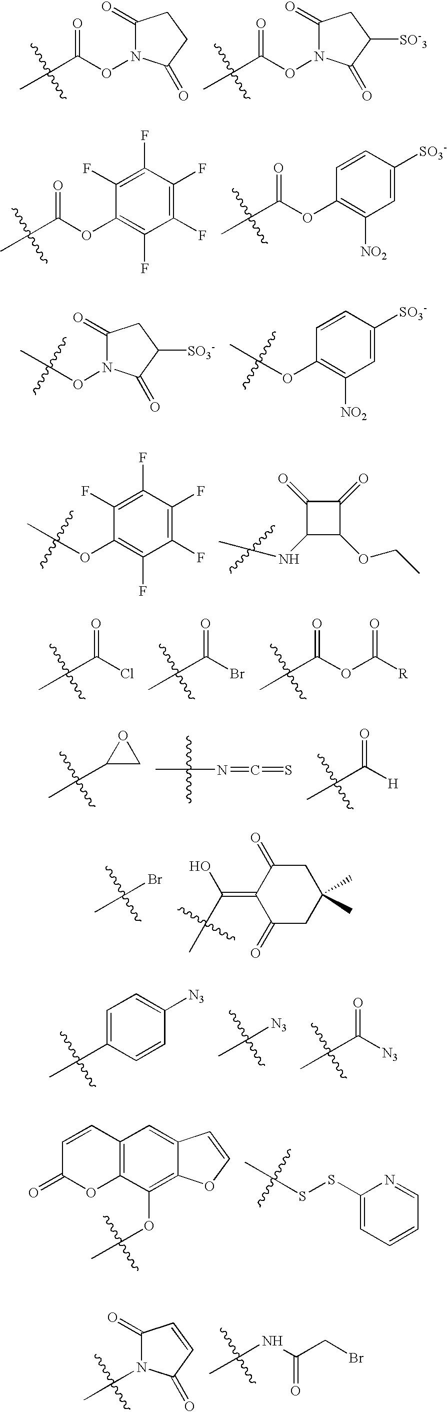

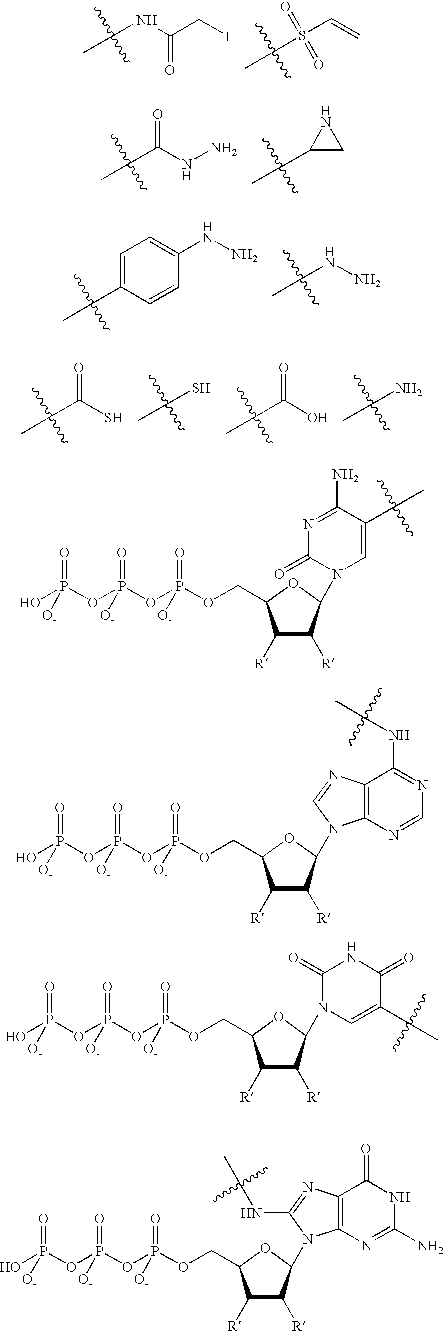

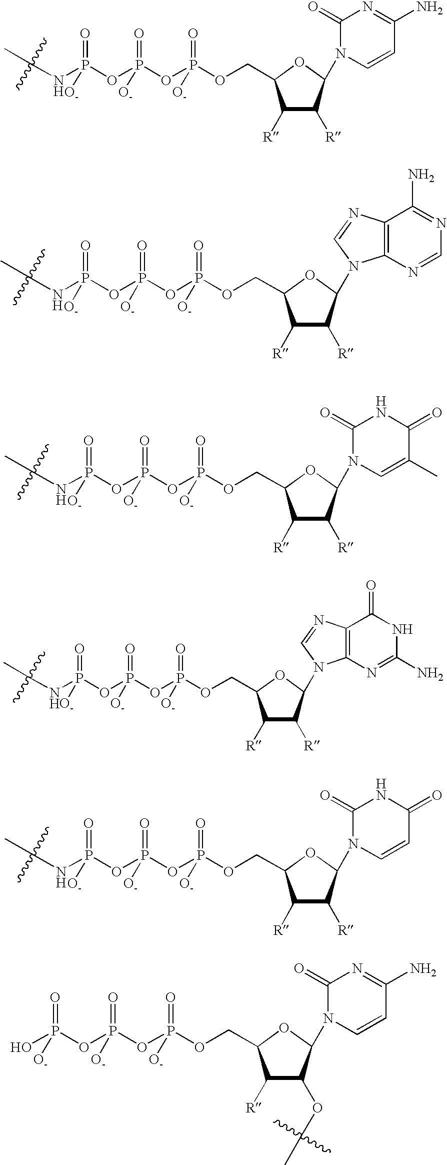

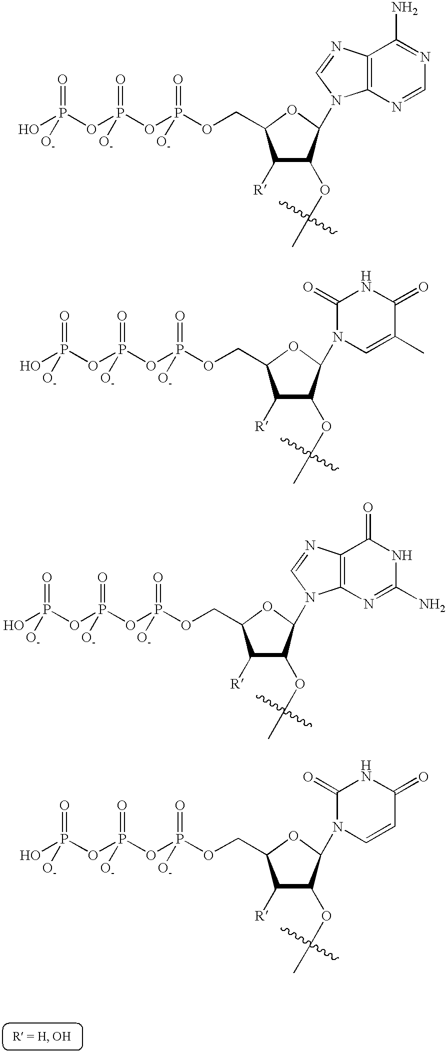

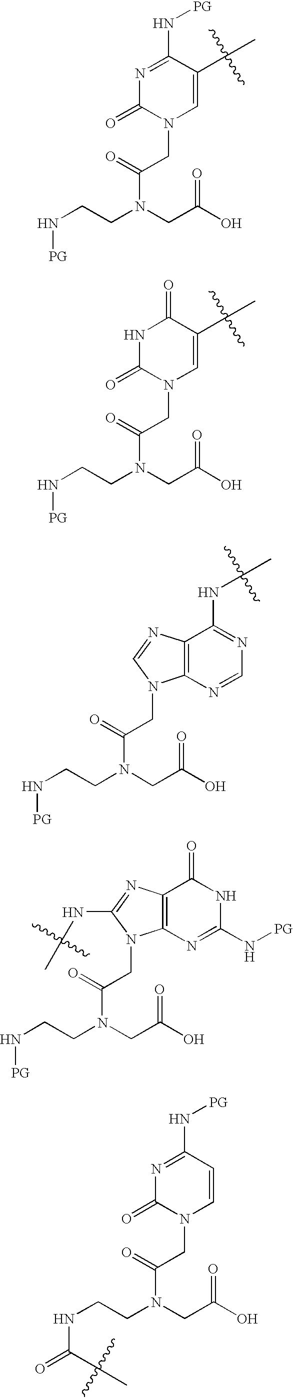

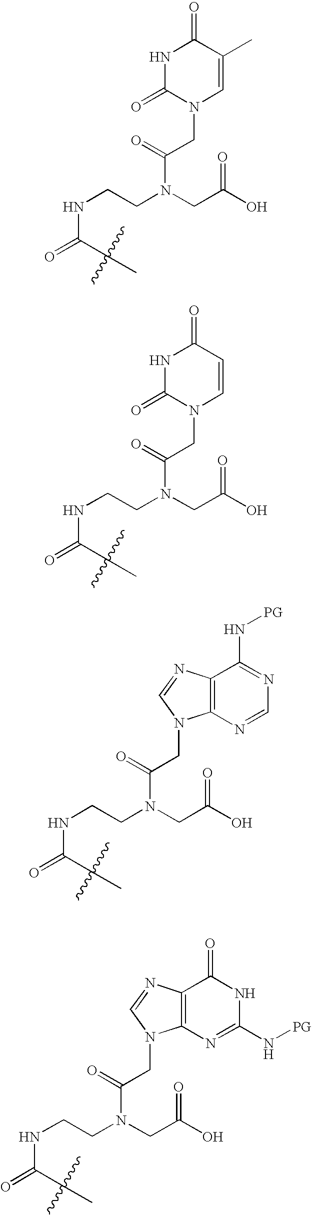

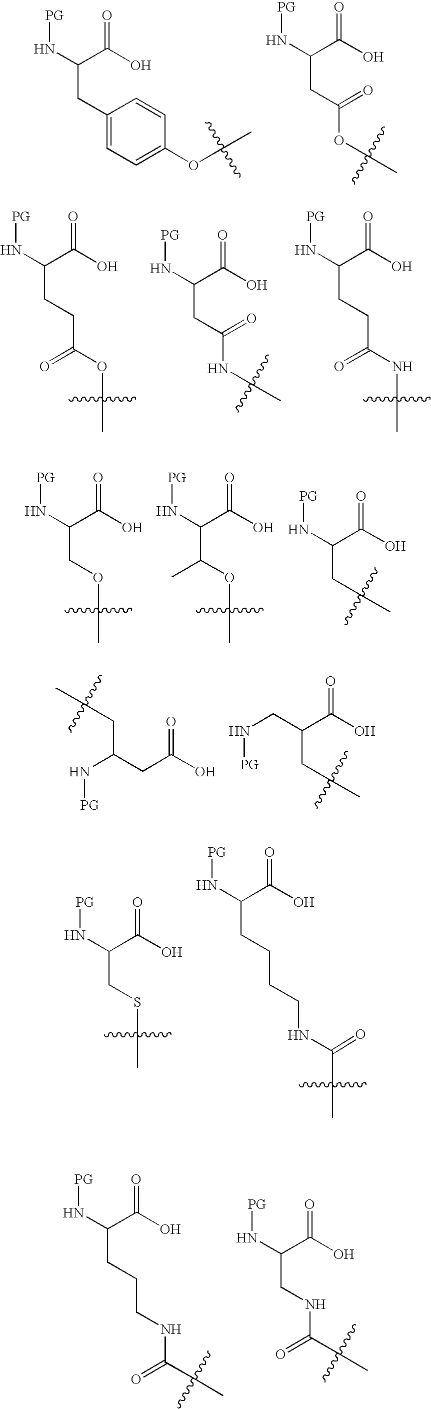

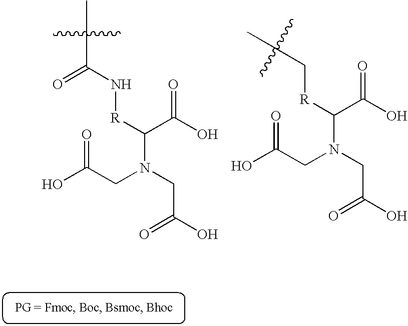

wherein R are independently from each other identical or different residues selected from the following group:
—H, linear, branched or cyclic alkyl residue or alkoxy residue comprising 1 to 15 carbon atoms, linear or branched alkenyl residue comprising 2 to 15 carbon atoms, protected or unprotected amine,
the kits further comprising polyclonal antibodies, monoclonal antibodies, or fragments thereof that are suitable to bind to the labelling compound.

Preferably the antibodies are monoclonal antibodies produced by a hybridoma obtainable according to the method of Franek et al. (Franek M, Kolar V, Granatova M, Nevorankova Z (1994). Monoclonal ELISA for 2,4-Dichlorophenoxyacetic Acid: Characterization of Antibodies and Assay Optimization. J Agric Food Chem. 42:1369-1374.). Further included are antigen-binding antibody fragments, e.g. monovalent Fab and divalent (Fab)₂, which are derivable from said monoclonal anti-2,4-D-antibodies.

The following compounds are included according to the invention (n=1, 2, 3, . . . , 13):

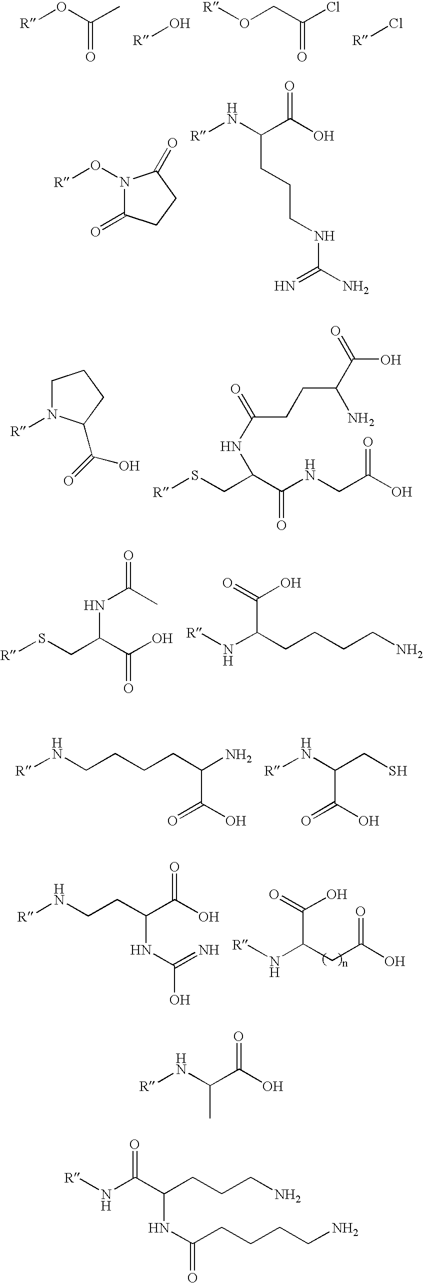

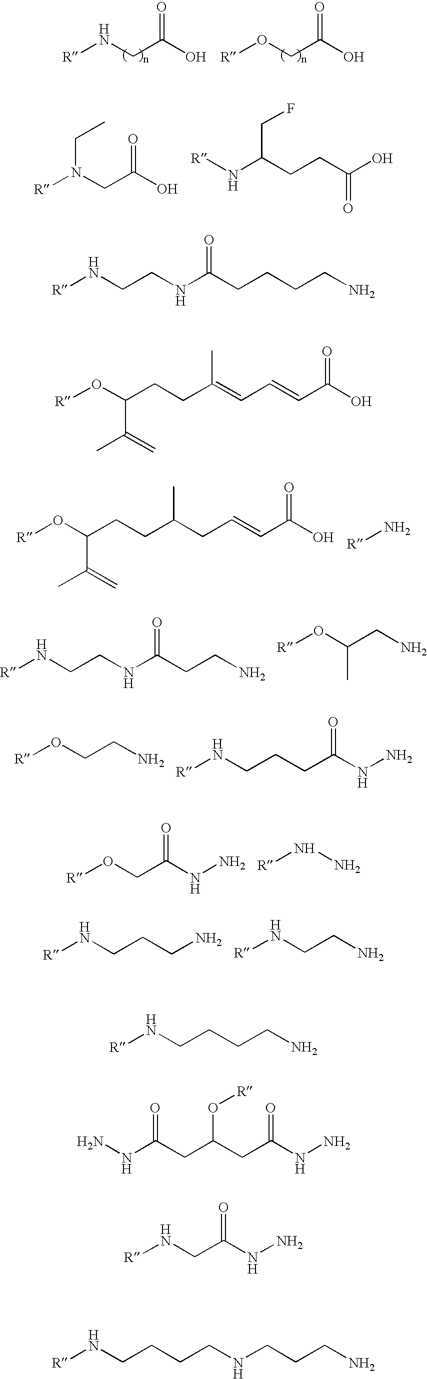

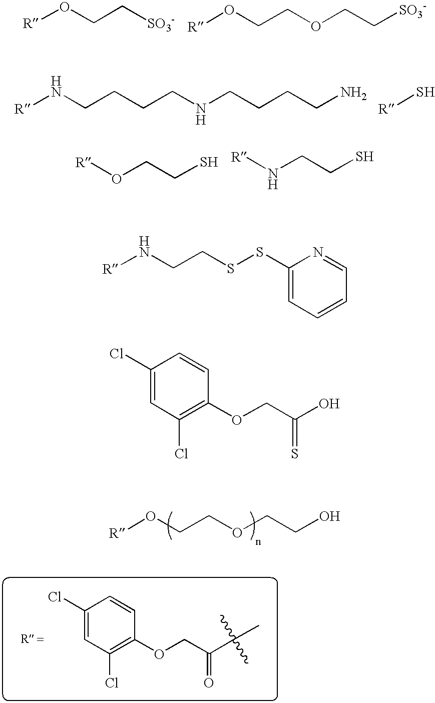

Wavy lines in the above structures indicate, that the marked bonds do not depict alkyl residues such as methyl groups which are often presented this way in chemical drawings in the scientific community.

Possible amino acids comprise all natural amino acids, preferably alanine, beta-alanine, aspartic acid, asparagine, arginine, citrulline, cysteine, glycine, glutamic acid, glutamine, histidine, homoserine, hydroxyproline, isoleucine, leucine, lysine, methionine, ornithine, phenylalanine, proline, sarcosine, serine, threonine, tryptophane, tyrosine and valine, each in L- and D-configuration, as well as non-natural amino acids, e.g. 2-aminoethylglycine, phenylglycine, penicillamine, norvaline, norleucine, alpha-aminobutyric acid, diaminopropionic acid, cyclohexylalanine, butylglycine, aminoisobutyric acid, thienylalanine, statine, each in L- and D-configuration, and aminooligoethyleneglycol-carboxylic acids and aminooligopropyleneglycol-carboxylic acids.

Protected amino acids comprise amino acids carrying a protecting group, e.g. S-acetamidomethyl- (Acm), t-butyloxycarbonyl- (Boc), t-butyl (tBu), trityl- (Trt), 2,2,4,6,7-pentamethyldihydrobenzofurane-5-sulfonyl- (Pbf), tosyl- (Ts), fluorenylmethoxycarbonyl- (Fmoc), (1,1,-dioxobenzo[b]thiophene-2-yl-methyl)oxycarbonyl- (Bsmoc), benzyloxycarbonyl- (CBz), benzhydryloxycarbonyl- (Bhoc) or beta-2-adamantyl- (Ada). Unprotected amino acids are thus amino acids carrying no such protecting group.

Nucleosides can be cytidine, uridine, thymidine, adenosine, guanosine in their syn- and anti-configurations and their mono-, di-, and triphosphates (nucleotides) and derivatives thereof, e.g. their cyclic forms, such as 3′-5′-cyclic adenosine monophosphate (cAMP), or their deoxy- or dideoxy-forms, or synthetic nucleoside analogues, such as xanthosine, hypoxanthosine, 6-mercaptopurine, 5-fluorouracil, 5-iodo-2′-deoxyuridine, 6-thioguanine, azothymidine or dideoxyinosine and their mono-, di-, and triphosphates (synthetic nucleotide analogues).

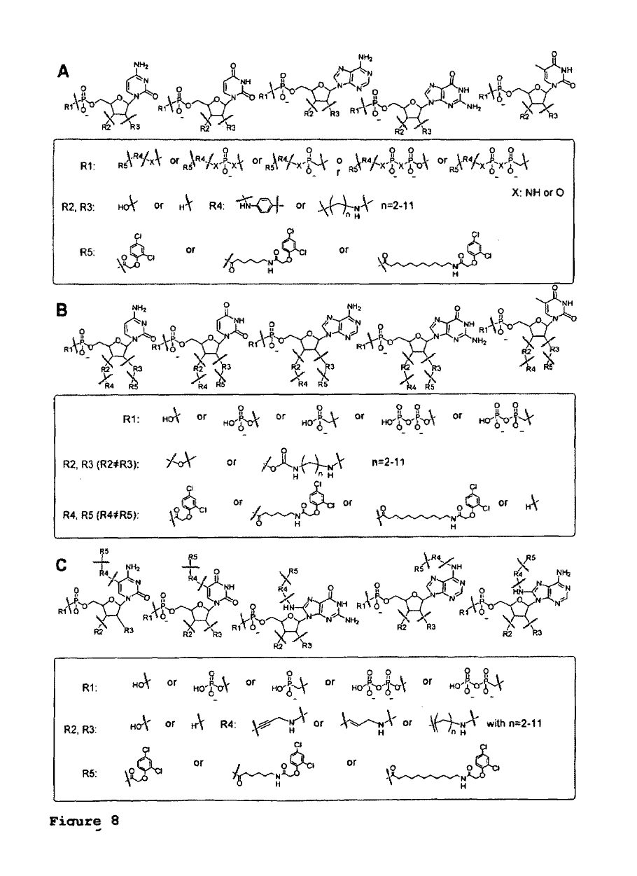

According to a particular embodiment of the invention instead of nucleotide-triphosphate-, deoxynucleotide-triphosphate- or dideoxynucleotide-triphosphate-residues in formula (I), the respective nucleotide residues mentioned in general description 5 (cf. examples) and the nucleotide derivatives which are available from the substituent combinations derivable from FIG. 8 (A), (B) and (C) can be used as well and are explicitly included herewith.

Saccharide moieties in the spacer are for example linear or branched oligosaccharides containing 1 to 10 equal or different monosaccharides, such as glucose, mannose, galactose, ribose, arabinose, N-acetylglucosamine or fructose, or containing 1 to 5 equal or different disaccharides, such as cellobiose, lactose, chitobiose or lactosamine, preferentially but not necessarily linked via beta-1,4-glycosidic bonds, or containing combinations or derivatives thereof. The spacer can furthermore contain or consist of O-glycosylated serine, threonine or N-glycosylated aspartic and/or glutamic acid. In addition the spacer can contain linear or branched polyoles with 3 to 15 hydroxyl-groups, which can be linked to any of the spacer moieties, such as saccharide- or polyol-structures, described above. According to one embodiment, the polyols as such can be used as spacers, i.e. without additional spacer components. Alditols, such as glucitol, mannitol, allitol oder galactitol, or cyclitol derivatives, such as derivatives of inositol, are examples of the polyols which are preferred according to the present invention.

The term “label mediating groups” (MVG), as used herein, relates to functional units, which mediate the labelling of substrate-molecules with 2,4-dichlorophenoxyacetyl moieties (2,4-D) or with 2,4-D-derivatives, which are equipped with spacers. Labelling of substrate-molecules with 2,4-D can either occur directly by a chemical reaction of the “label mediating group” with the substrate-molecule, whereby 2,4-D or the 2,4-D derivative is transferred onto the substrate molecule, or by incorporation of the “label mediating group”, which in this case remains covalently linked to the 2,4-D moiety, into a respective substrate-molecule. In the former case the “label mediating group” is also referred to below as “reactive group” (RG). “Label mediating groups” which allow direct labelling of substrate-molecules are e.g. amine-, thiol-, aldehyde-reactive groups or groups having multiple reactivities. “Label mediating groups” which enable indirect labelling with 2,4-D are e.g. protected or unprotected amino acids, nucleotides, saccharides or lipids.

In the context of the present invention ‘linear or branched alkyl residue’ is a hydrocarbon-residue, described by the formula CₙH₂ₙ₊₁ and CₙH₂ₙ, respectively. In particular residues comprising 1, 2, 3, 4, 5 . . . 14 or 15 carbon atoms are included. Explicitly included are methyl(-ene), ethyl(-ene), propyl(-ene), isopropyl(-ene), butyl(-ene), isobutyl(-ene), sec. butyl(-ene), tert. butyl(-ene), pentyl(-ene), isopentyl(-ene), neopentyl(-ene), hexyl(-ene), heptyl(-ene), octyl (-ene), nonyl(-ene), decyl(-ene), and methyl-, ethyl- or propyl-substituted heptyl(-ene)-, octyl(-ene)-, nonyl(-ene)- or decyl(-ene)-moieties, such as 6,7,8,9,10-pentamethyldecyl(-ene) or 8-methyl-9,10-diethyldecyl(-ene).

Non-limiting examples for a linear or branched alkenyl residue are hydrocarbon residues comprising 2, 3, 4, 5, . . . , 14 or 15 carbon atoms as well as one or, if applicable, more than one cis or trans configurated C—C-double bonds. Explicitly included are propenyl(-ene), 2-butenyl(-ene), 3-butenyl(-ene), 2-pentenyl(-ene), 3-pentenyl(-ene), 1,3-hexadienyl(-ene), 1,5-hexadienyl(-ene), alkenyl residues with conjugated C—C-double bonds, such as 1,3,5,7,9-decapentenyl(-ene).

Alkoxy residues mentioned above include linear or branched alkyl-residues as defined above, that are bound, via an oxygen-atom, to the C— atoms in the formulas shown above.

The amine mentioned above can be a primary, secondary or tertiary amino group: —NH₂, NHR′ or NR′₂, wherein R′ can be an alkyl residue as defined above.

Preferred compounds are defined by formula (II)

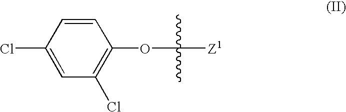

wherein Z1 is selected from the following substituents:

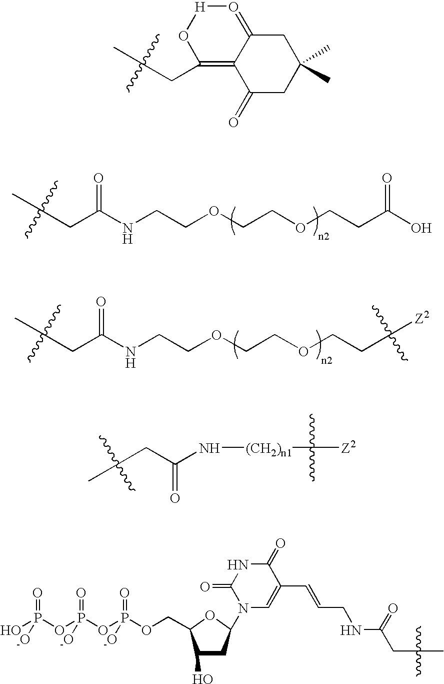

where n1 is 1 to 15,
wherein Z2 is selected from the following substituents:

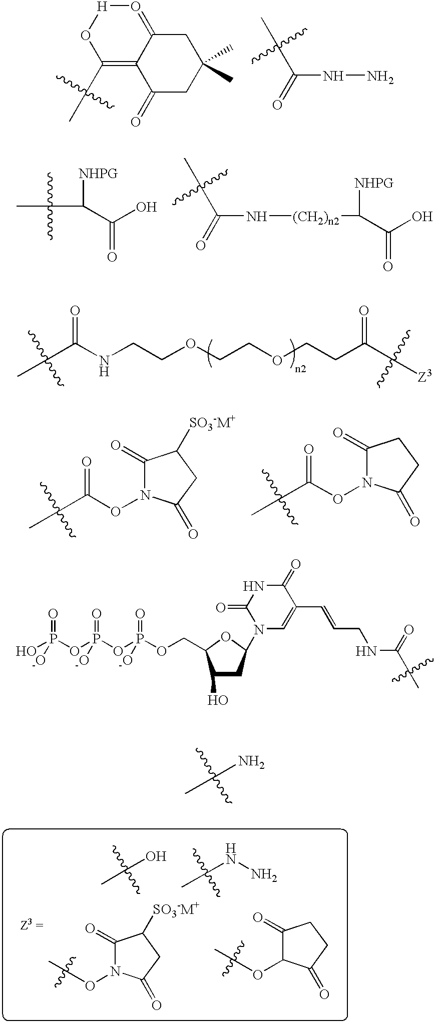

where n2 is 1 to 15,
wherein M⁺ is a monovalent, inorganic or organic cation,
wherein PG is an amino-protecting group.

According to the preceding definition n2 can be an integer in the range of 1 to 15, thus 1, 2, 3, 4, . . . , 14 or 15. Particularly preferred are compounds where n1 is 5 or n1 is 10 and/or is 4 or n2 is 11.

Cations concerned in particular are lithium, potassium, ammonium, rubidium, caesium, preferably sodium or tetraalkylammonium (in particular tetramethylammonium, tetraethylammonium, tetrapropylammonium, tetrabutylammonium, tetrapentylammonium, or tetrahexylammonium) ions.

Preferred amino-protecting groups are, for example, (1,1,-dioxobenzo[b]thiophene-2-yl-methyl)oxycarbonyl (Bsmoc), t-butyloxycarbonyl (Boc) or benzyloxycarbonyl (CBz) and in particular fluorenylmethoxycarbonyl (Fmoc).

Instead of the deoxyuridin-5′-triphosphate residue, the respective nucleotide residues mentioned in general description 5 (cf. examples) and the nucleotide derivatives which are available from the various substituent combinations derivable from FIG. 8 (A), (B) and (C) can be used as well and are explicitly included herewith.

According to a particular embodiment of the invention kits comprising compounds defined by formulas (III) and (IV) are provided:

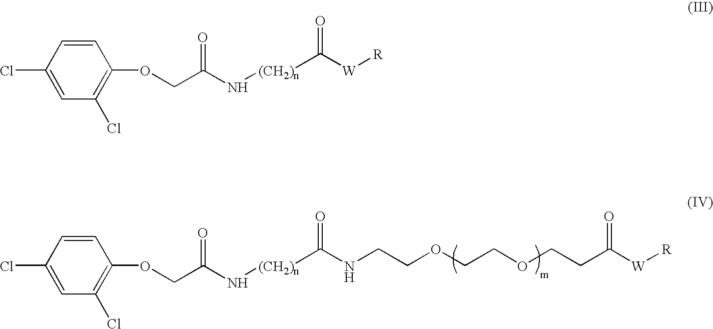

where n is 1, 2, 3, 4, 5, 6, 7, 8, 9, 10, 11, 12, 13, 14, 15, and where m is 1, 2, 3, 4, 5, 6, 7, 8, 9, 10, 11, 12, 13, 14, 15.

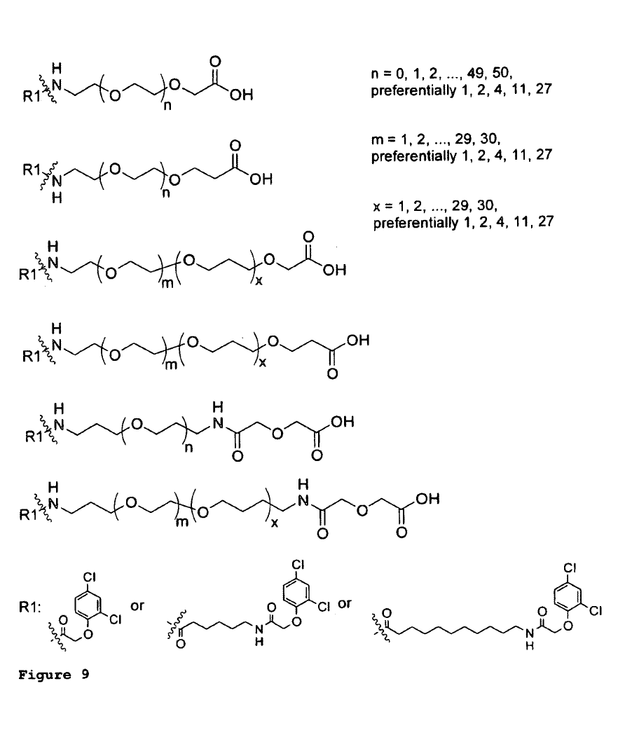

The residue W is preferably —O— or —NH—, and R is preferably hydrogen (—H), succinimidyl, sulfo-succinimidyl, amino (—NH₂), 5-(allyl)-2′-deoxyuridine-5′-triphosphatidyl or Fmoc-lysinyl. Instead of the 5-(3-amidoallyl)-2′-deoxyuridin-5′-triphosphatidyl derivative, the respective nucleotide derivatives mentioned in general description 5 (cf. examples) and possible nucleotide derivatives which are available from the various substituent combinations derivable from FIG. 8 (A), (B) and (C) can be used as well and are explicitly included herewith, as are the amino acid derivatives described in general description 4 and general description 6. Instead of the derivatives of general formula (IV)—and explicitely included according to the present invention—derivatives which are derivable from the polyethylene glycol derivatives of FIG. 9, can be used as well.

Compounds of the kits of the invention are derivatives of 2,4-dichlorophenoxyacetate (2,4-D), herein also designated “2,4-D-derivatives”. 2,4-dichlorophenoxyacetate as well as ester, amide or other derivatives and salts thereof were originally developed and used as herbicides. Conjugates, in which 2,4-dichlorophenoxyacetate is covalently linked to low- or high-molecular weight carriers, are used for a number of bioanalytical purposes. Mainly, such derivatives are used in competitive immunoassays, employed for the toxicological analysis of the herbicide in drinking water, in food, or in body fluids. (See e.g.: Kroger S, Setford S J, Turner A P (1998). Immunosensor for 2,4-dichlorophenoxyacetic acid in aqueous/organic solvent soil extracts. Anal Chem. 70:5047-5053; Bier F F, Kleinjung F, Ehrentreich-Forster E, Scheller F W (1999). Changing functionality of surfaces by directed self-assembly using oligonucleotides-the Oligo-Tag. Biotechniques. 27:752-756; Halamek J, Hepel M, Skladal P (2001). Investigation of highly sensitive piezoelectric immunosensors for 2,4-dichlorophenoxyacetic acid. Biosens Bioelectron. 16:253-260;). In the assays described 2,4-D is coupled directly in situ to carrier molecules by standard methods and is detected in competition with free 2,4-D by specific antibodies. (see: Fránek M, Kolar V, Granatova M, Nevorankova Z (1994). Monoclonal ELISA for 2,4-Dichlorophenoxyacetic Acid: Characterization of antibodies and assay optimization. J Agric Food Chem. 42:1369-1374; Fránek M, Brichta J (2003). Identification of Monoclonal Antibodies against 2,4-D Herbicide by ELISA and DNA Sequencing. J Agric Food Chem. 51:6091-6097.). Antibodies required for this were obtained by immunizing test-animals with 2,4-D-protein- or 2,4-D-poly-L-lysin-conjugates. (see e.g.: Fránek M (1989). CS8707109A1, Immunogenes for production of antibodies against polychlorinated biphenyls and method of their preparation; Schecklies E (1995). DE19529250C1, Preparation of hapten-carrier conjugates containing polyaminoacids as carriers.).

Compounds of the kits of the invention are very suitable to be bound to carrier molecules, solid phases and substrate molecules (in the following also termed “substrates”). Carrier molecules and substrate molecules are soluble substances, and solid phases are insoluble substances, to which 2,4-dichlorophenoxyacetate derivatives can be linked via the “MVG” moiety. Substrate molecules are, e.g. macromolecules (macromolecular substrate molecules), such as biogenous macromolecules. Wherein macromolecules are chemical compounds with a molecular mass of at least 500 g/mol. Biogenous macromolecules are chemical compounds whose structure corresponds to compounds resulting from or involved in metabolic processes. However, biogenous macromolecules as defined in the context of this invention do not have to be the result of metabolic processes but can be of different origin.

The terms “soluble” and “insoluble” refer to aqueous or organic liquids, respectively.

Preferably the linkage between the substrates and the compounds of the present invention is a covalent bond, however, other linkages may be suitable as well and are thus explicitly included.

In the context of the invention “stable in water” means that approximately 50% of the amount of the corresponding compound are still reactive in fluid media having a water content of at least 80%, and a pH value in the range of 3 to 9 and at a temperature of 0° C. up to a few minutes, preferably 5 minutes, or less than 50% of the amount of the corresponding compound decomposes in fluid media having a water content of at least 80%, and a pH value of 7 and at 4° C. in the course of 20 minutes, preferably in the course of 1 hour, respectively.

“Soluble in water” means in context of the invention that 100 μMole of the corresponding 2,4-dichlorophenoxyacetate derivatives of the invention are soluble in a volume of 1 l of a fluid medium containing at least 80% water at 25° C.

2,4-dichlorophenoxyacetate is a hapten. The term hapten describes a small organic molecule that is a specific ligand for an antibody which is vice versa specific for this hapten. Thus, regions of the substrate molecule proximal to substrate-bound 2,4-dichlorophenoxyacetate residues could disturb binding. On the other hand, the antibody might also recognize structural elements of the hapten-carrier conjugate against which it was raised, in particular the structure of the linkage between hapten and carrier molecule, together with the hapten, and therefore require such carrier molecule for high affine binding. Compounds of the kits claimed here comprise spacers to reduce disturbing effects and/or to imitate the linkage structure of the hapten-carrier-conjugate.

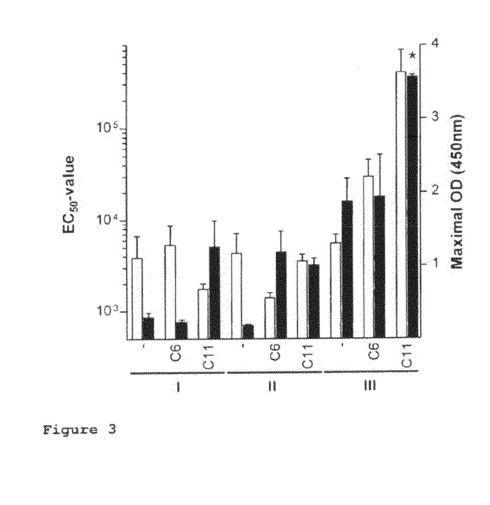

If 2,4-dichlorophenoxyacetate derivatives comprise aliphatic spacers, e.g. 11-aminoundecanoic acid or 6-aminohexanoic acid, the lower detection limit of substrates labelled therewith is better than that of substrates labelled with 2,4-dichlorophenoxyacetate derivatives that contain no spacer when the assay is performed with the antibodies used in this invention. The affinity of the binding between the 2,4-dichlorophenoxyacetate group and the antibody is increased in particular when linear alkyl spacers are linked to the carboxyl-group of 2,4-dichlorophenoxyacetate. 2,4-Dichlorophenoxyacetate derivatives of the present invention include preferentially linear alkyl spacers without or in combination with oligoethyleneglycol substructures according to the formulas III and IV wherein n and m are independently from each other integers in the range of 1-15. The benefit of spacers, comprising aminohexanoic acid (n=5) or 11-aminoundecanoic acid (n=10), in an enzyme-linked immunosorbent assay (ELISA) is exemplarily shown in FIG. 3. 2,4-Dichlorophenoxyacetate derivatives of the present invention can additionally include residues in the spacers or the “label mediating groups” (MVG) according to formula I that increase the solubility in water. These residues can be composed of charged or oligoethyleneglycol or oligopropyleneglycol moieties. Saccharide moieties in the spacer which increase solubility are for example linear or branched oligosaccharides containing 1 to 10 equal or different monosaccharides, such as glucose, mannose, galactose, ribose, arabinose, N-acetylglucosamine or fructose, or containing 1 to 5 equal or different disaccharides, such as cellobiose, lactose, chitobiose or lactosamine, preferentially but not necessarily linked via beta-1,4-glycosidic bonds, or containing combinations or derivatives thereof. The spacer can furthermore contain or consist of O-glycosylated serine, threonine or N-glycosylated aspartic and/or glutamic acid. In addition the spacer can contain linear or branched polyoles with 3 to 15 hydroxyl-groups, which can be linked to the spacer moieties, such as the saccharide- or polyol-structures described above. Such saccharide moieties in the spacer can be substituted on one side with 2,4-dichlorophenoxyacetic acid or a 2,4-D derivative including an alkyl spacer, on the other side with one of the “label mediating groups” (MVG) defined for the generic formula I. An “increase in solubility” means in context of the invention that the corresponding 2,4-dichlorophenoxyacetate derivatives are soluble in fluid media containing at least 80% water.

Charged moieties in substitutents of the spacer or MVG of the generic formula I can be for example: carboxylic acid-, sulfonic acid- or phosphate-groups as negatively charged moieties as well as ammonium- or guanidyl-groups as positively charged moieties.

Oligoethyleneglycol moieties are for example the following structures: linear or branched ethylenglycol homopolymers, or propyleneglycol homopolymers, as well as mixed ethyleneglycol/propyleneglycol copolymers with mean molecular weights of 100 to 5000 g/mole which are substituted on one or more sites. Explicitly included are 1-ethoxy-2-ethoxy-ethyl, 1-ethoxy-2-(2-ethoxyethoxy)ethyl, 1-ethoxy-2-[2-(2-ethoxyethoxy)ethoxy]ethyl, and homologues thereof substituted on one side with 2,4-dichlorophenoxyacetic acid or a 2,4-D derivative including an alkyl spacer, on the other side with one of the “label mediating groups” (MVG) defined for the generic formula I.

Coupling of the compounds of the kits of the invention to substrates allows them to be used as markers. The linkage between substrate and 2,4-dichlorophenoxyacetate derivative is mediated by the MVG moiety, according to the invention. Some groups (RG) can be coupled to substrates without in situ activation, in other cases in situ activation is necessary. Amino-reactive 2,4-dichlorophenoxyacetate derivatives, such as aldehydes, aziridines or epoxydes, as well as active esters, like pentafluorophenyl-, succinimidyl- or sulfosuccinimidylesters can be coupled directly to amino groups without further activation. Carboxylates in aqueous solutions have to be activated by addition of e.g. N-(3-dimethylaminopropyl)-N′ethylcarbodiimide-hydrochloride (EDAC), in order to be coupled to substrates. Aldehydereactive 2,4-dichlorophenoxyacetate derivatives, such as hydrazides, are not further activated in situ, as well as sulfhydryl-reactive derivatives, such as maleimides, vinylsulfones or pyridylthiols.

Kits of the invention further comprise polyclonal antibodies, monoclonal antibodies (mAbs), or fragments thereof, preferably specific mAbs, that bind to the 2,4-dichlorophenoxyacetate derivatives of the invention. Such antibodies are obtainable by using conjugates of 2,4-D-derivatives and substrate molecules, such as proteins or glycoproteins. Antibodies concerned in particular are the monoclonal antibodies derived from hybridoma clones, such as E2/G2, E2/B5, E4/C2, G5/E10, F6/C10, B5/C3, and B7, respectively, which are obtainable according to the method described in detail in the literature (see: Fránek M (1989) CS8707109A1. Immunogenes for production of antibodies against polychlorinated biphenyls and method of their preparation; Fránek M, Kolar V, Granatova M, Nevorankova Z (1994). Monoclonal ELISA for 2,4-Dichlorophenoxyacetic Acid: Characterization of Antibodies and Assay Optimization. J Agric Food Chem. 42:1369-1374.).

The aminoalkyl-derivatives of 2,4-D described herein utilize a so called bridge-binding effect which all of the 2,4-D-specific antibodies used in this invention display (see Table 4; e.g. Eremin S A, Lunskaya I M, Egorov A M (1993). The influence structure of labelled antigen on sensitivity and specificity for polarisation fluoroimmunoassay of 2,4-dichlorophenoxyacetic acid (2,4-D). Russian Journal of Bioorganic Chemistry 19:836-843).

Thus, mAbs suitable within the context of the present invention besides the antibodies produced by the above-referenced hybridomas include, but are not limited to 2,4-D specific monoclonal antibodies which also exert the so called bridge-binding effect mentioned above.

The 2,4-D derivatives of the present invention are highly suitable labelling molecules as due to the presence of the “Spacer” moiety, a lower detection limit is observed when using 2,4-D-specific antibodies as compared to compounds lacking the “Spacer” and thus not allowing the bridge-binding effect mentioned above. For example, if 2,4-D is equipped with an aliphatic linker between the 2,4-D moiety and MVG, like 11-aminoundecanoic acid or 6-aminohexanoic acid, the detection limit of biomolecules labelled therewith is lower as compared to biomolecules labelled with 2,4-D not having any linker between the 2,4-D moiety and MVG when the assay is performed with the antibodies used in this invention.

The Kits described herein comprise the labelling compounds of the invention that are suitable to be linked to substrates in the course of a labelling procedure that results in labelled substrates. Labelled substrates can be detected with antibodies of the invention in the course of detection assays. Depending on the substrate and the “MVG” moiety, labelling procedures can be performed in different buffers and/or solutions using different reagents. Furthermore, detection assays can be performed in a range of different buffers and/or solutions.

In one embodiment of the present invention, the kits comprise buffers, solutions, means and reagents suitable to perform labelling procedures as well as detection assays.

The following exemplary embodiments of the kit shall not limit the invention. Furthermore the mentioned kits or constituents thereof can be combined or assorted in a different manner.

For labelling of proteins, glycoproteins, peptides, nucleosides, nucleotides, saccharides or lipids the kit may comprise 2,4-D derivatives of the present invention, anhydrous organic solvent, such as dimethylsulfoxide or N,N-dimethylformamide (DMF), sodium m-periodate, glycine, hydrazine hydrate, sodium acetate, sodium tetraborate, bicarbonate or Dulbecco's phospate-buffered saline for labelling of substrates, control substances, such as glycosylated and non-glycosylated proteins, as well as semi-permeable dialysis membranes and gel filtration columns, such as Sephadex G-25, for purification of the labelled conjugates, as well as 2,4-D labelled standard, such as albumin or immunoglobulin, and preservatives, such as sodium azide or thimerosal.

For labelling of DNA or PCR-fragments or generation of DNA-hybridization probes the kit may comprise an appropriate DNA-polymerase, such as Taq DNA polymerase, dideoxy- or deoxynucleotides, such as 2′-deoxyadenosine 5′-triphosphate (dATP), 2′-deoxycytidine 5′-triphosphate (dCTP), 2′-deoxyguanosine 5′-triphosphate (dGTP), 2′-deoxythymidine 5′-triphosphate (dTTP), 2′-deoxyuridine 5′-triphosphate (dUTP) and labelled dideoxy- or deoxynucleotides, such as 2,4-D-11-dUTP, buffer with and without magnesium chloride (MgCl₂), such as potassium chloride and tris containing buffers, MgCl₂-stock solution, control oligonucleotides specific for a control template, control template DNA, 2,4-D labelled control DNA and sterile water. Additionally, 2,4-D labelled DNA molecular weight markers can be included.

For labelling of RNA-fragments or generation of RNA-hybridization probes the kit may comprise an appropriate transcription vector containing a polylinker site and promoters of appropriate RNA polymerases, such as SP6 and T7, an appropriate RNA polymerase, such as SP6 or T7, 2,4-D labelled control RNA, unlabelled control RNA, control DNA, nucleotides, such as adenosine 5′-triphosphate (ATP), cytidine 5′-triphosphate (CTP), guanosine 5′-triphosphate (GTP), thymidine 5′-triphosphate (TTP), uridine 5′-triphosphate (UTP) and labelled nucleotides, such as 2,4-D-11-UTP, a RNase free DNase, a RNase inhibitor and optionally 2,4-D labelled RNA molecular weight markers.

For the discrimination of carbohydrate moieties, such as glycans (polysaccharides) linked to glycoproteins or glycoconjugates, the kit may comprise 2,4-D labelled lectins, such as wheat germ agglutinin, peanut agglutinin or sambucus nigra agglutinin, control glycoconjugates, such as the protein transferrin, carrying polysaccharides, which bind to the included 2,4-D labelled lectins, and non-glycosylated control proteins. For labelling of biological or artificial membranes, such as phospholipid bilayers (e.g. plasma membranes and intracellular membranes of live cells, or small unilamellar vesicles and liposomes), the kit may comprise 2,4-D labelled lipids, such as phospholipids (including phospholipid analogues such as phosphatidylinositol), sphingolipids (including gangliosides and ceramides), fatty acids, triglycerides or steroids, stabilizing additives, such as bovine serum albumin, organic solvents, such as ethanol or chloroform, Hanks' buffered saline, phosphate-buffered saline, tris, 4-(2-hydroxyethyl)-1-piperazineethanesulfonic acid (HEPES), glutaraldehyde, paraformaldehyde, sterile deionized water, and preservatives, such as sodium azide or thimerosal.

For the detection of labelled conjugates the kit may comprise 2,4-D labelled protein molecular weight marker, anti-2,4-D antibodies or antigen binding fragments thereof referred to herein, biomolecule binding supports such as nitrocellulose, polyvinylidene fluoride (PVDF), nylon or cationized nylon membranes, blocking reagents, such as casein, synthetic detergents or non-fat dry milk, 2,4-D labelled standard, such as albumin or immunoglobulin, preservatives, such as sodium azide or thimerosal. The anti-2,4-D antibodies can be provided unlabelled or labelled with dyes, such as fluorescein, rhodamine or AlexaFluor680, or enzymes, such as horseradish peroxidase, alkaline phosphatase or beta-galactosidase, for chemiluminescent, colorimetric or fluorescent detection of the substrate-bound 2,4-D. In case of providing unlabelled anti-2,4-D antibodies, anti-mouse secondary antibodies labelled as described above can be included. Chromogenic substrates, such as ABTS (2,2′-azino-bis(3-ethylbenzothiazoline-6-sulfonic acid), TMB (3,3′,5,5′-tetramethylbenzidine), or NBT/BCIP (3,3′-(3,3′-dimethoxy-4,4′-biphenylene)-bis-[2-(4-nitrophenyl)-5-phenyl-2H-tetrazolium blue chloride] (4-Nitro blue tetrazolium chloride) and 5-Bromo-4-chloro-3-indolylphosphate) can be included in the kit.

The invention further relates to the use of the kits for the detection of substrate molecules (see above).

In particular, the invention relates to the use of the kits of the invention for the detection of amino acids, branched or unbranched oligopeptides, polypeptides, proteins, nucleic bases, nucleosides, nucleotides, oligonucleotides, polynucleotides, nucleic acids, monosaccharides, oligosaccharides, polysaccharides, glycoproteins, or lipids. Preferably the invention is concerned with the detection of amino acids, polypeptides of 2 to 50 amino acids, proteins having a molecular mass of more than 5 kDa, nucleic bases, nucleosides or nucleotides and derivatives thereof, such as their mono-, di- or triphosphates and cyclic forms, polynucleotides of 2-100 nucleotides, nucleic acids with a length of 100-5000 nucleotides, saccharides, oligosaccharides of 2 to 30 monosaccharides or polysaccharides having a molecular mass of more than 5 kDa.

Saccharides comprising 1 to 30 monosaccharide moieties may contain the following aldoses and ketoses: D-erythrose, D-threose, D-ribose, L-arabinose, D-xylose, D-lyxose, D-allose, D-altrose, D-glucose, D-mannose, D-gulose, D-idose, D-galactose, D-talose and D-tetrulose, D-ribulose, D-xylulose, D-psicose, D-fructose, D-sorbose, D-tagatose as well as D-glucosamine, N-acetyl-D-glucosamine (GlcNAc), D-galactosamine, N-acetyl-D-galactosamine (GalNAc), D-mannosamine, N-acetyl-D-mannosamine (ManNAc), L-daunosamine, N-acetyl-daunosamine, L-fucose, L-rhamnose, D-quinovose, D-olivose, D-digitoxose, D-cymarose, D-glucuronic acid, D-mannuronic acid, D-galacturonic acid, L-iduronic acid, 2-deoxy-D-ribose, N-acetyl-muraminic acid (MurAc), 5-N-acetyl-neuraminic acid (Neu5Ac) and 3-deoxy-D-manno-oct-2-ulopyranosonic acid (Kdo).

Nucleic bases can be purine or pyrimidine bases, such as cytosine, uracil, thymine, adenine, guanine, xanthin or hypoxanthine or derivatives thereof.

Nucleosides can be cytidine, uridine, thymidine, adenosine, guanosine in their syn- and anti-configurations and their mono-, di-, and triphosphates (nucleotides) and derivatives thereof, e.g. their cyclic forms, such as 3′-5′-cyclic adenosine monophosphate (cAMP), or their deoxy- or dideoxy-forms, or synthetic nucleoside analogues, such as xanthosine, hypoxanthosine, 6-mercaptopurine, 5-fluorouracil, 5-iodo-2′-deoxyuridine, 6-thioguanine, azothymidine or dideoxyinosine and their mono-, di-, and triphosphates (synthetic nucleotide analogues).

Lipids can be either cyclic, acyclic or polycyclic and composed of either saturated or unsaturated fatty acids, such as butyric acid, dodecanoic acid or alpha-linolenic acid, or triglycerides, waxes, or membrane forming lipids, such as phospholipids, glycerophospholipids, glycolipids or sphingolipids (e.g. phosphatidylcholine, phosphatidylethanolamine, phosphatidylserine, phosphatidylinositol, diphosphatidyl glycerol, dioleolylphosphatidylethanolamine, sphingomyelines, cerebrosides, gangliosides, ceramides or sulfatides), or terpenoids, e.g. steroids, retinoids or carotinoids (e.g. cholesterol, phytosterol, ergosterol, vitamin D, digitalis, strophantin, testosterone or progesterone).

In the context of the present invention the term “assay” refers to a qualitative or quantitative detection method, measuring method or examination method for the detection or measurement of substrate molecules (analytes). This includes, in particular, immunological assays, solid-phase supported diagnostic applications, enzyme-linked immunosorbent assays, hybridization assays, polymerase chain reactions and biological labelling and biological uptake experiments.

In the context of the present invention the term “immunological assay” designates an assay where antibodies are used as ligands to bind an analyte. These assays include particularly enzyme-linked immunosorbent assays and hybridization assays, which shall not limit the invention. The term “hybridization assay” includes assays where probes labelled with 2,4-D derivatives of the present invention are used in biological standard assays, such as polymerase chain reactions, gene expression analyses or blot analyses.

In the context of the present invention the term “solid-phase supported diagnostic application” designates a diagnostic assay, in which capture molecules that are specific for an analyte are immobilized on a solid phase. Thereby an analyte can be selectively bound from fluid media and unbound components can be removed from the solid phase by washing. Subsequently, the analyte can be detected by using labelled probes.

In this context, analytes are the components to be analyzed. Analytes can be molecules such as substrate or carrier molecules defined in the context of the invention, as well as amino acids, branched or unbranched oligopeptides, polypeptides, proteins, nucleic bases, nucleosides, nucleotides, oligonucleotides, polynucleotides, nucleic acids, monosaccharides, oligosaccharides, polysaccharides, glycoproteins or lipids. Preferably, the oligo- and polypeptides comprise 2 to 50 amino acids, the polynucleotides comprise 2-100 nucleotides, the oligosaccharides comprise 2 to 30 monosaccharides. Polysaccharides and proteins preferably have a molecular mass of more than 5 kDa. Nucleic acids preferably encompass 100 to 5000 nucleotides. Additionally, analytes can be equipped with additional markers, such as biotin, enzymes, or dyes, enabling their detection with standard chemiluminescent, calorimetric or fluorescent methods.

The term “biological labelling experiment” designates in the context of the present invention an experiment, in which a substrate which is labelled with the 2,4-dichlorophenoxyacetate derivatives of the present invention is used to label biological probes. Such biological probes may include, but are not restricted to microorganisms, tissues, body fluids, living cells, plasma membranes and intracellular membranes, artificial membranes or liposomes, nuclei, organelles and subcellular structures, receptors and extracellular matrix components.

The term “biological uptake experiment” designates in the context of the present invention an experiment, in which a substrate which is labelled with the 2,4-dichlorophenoxyacetate derivatives of the present invention is administered to an animal in vivo either peripherally (e.g. by feeding) or systemically (e.g. by injection). After killing of the animal the destination of the labelled substrate in different cells or tissues is determined with specific 2,4-D antibodies of the present invention. Alternatively, a tissue explant or cultured cells are incubated with a substrate labelled with 2,4-D derivatives and the destination of the substrate in different cell types or subcellular compartments is determined with specific 2,4-D antibodies referred to herein.

The invention further relates to the use of the 2,4-D specific monoclonal antibodies referred to herein, suitable to detect substrate molecules by binding to the labelling compounds of the invention that are bound to the substrate molecules.

The present invention is described below by means of examples and figures, representing preferred embodiments of the invention, which however, shall not limit the invention.

### DESCRIPTION OF THE FIGURES

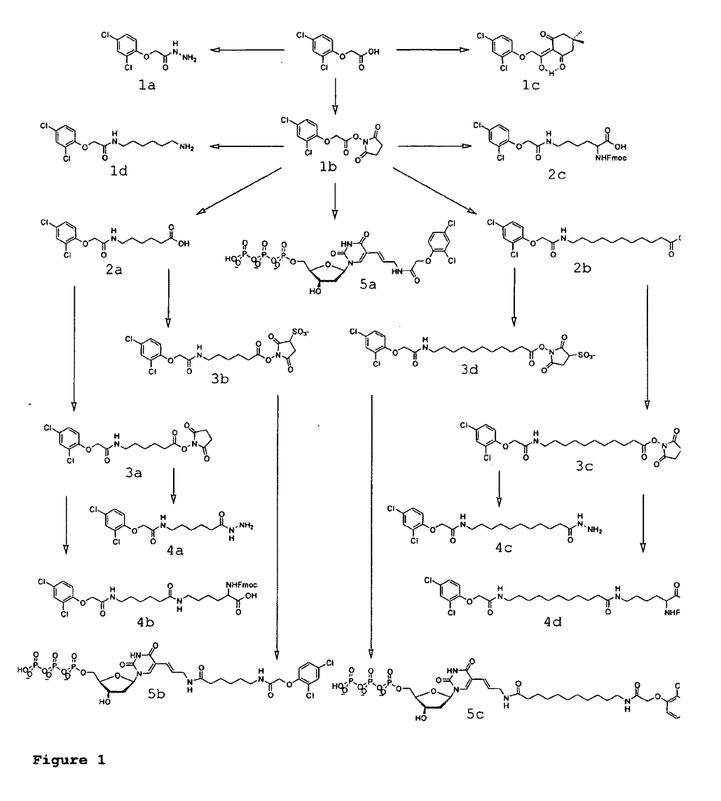

FIG. 1: Reaction scheme I.
Overview of the synthesis of some of the 2,4-dichlorophenoxyacetate derivatives of the kits of the invention described in the examples.

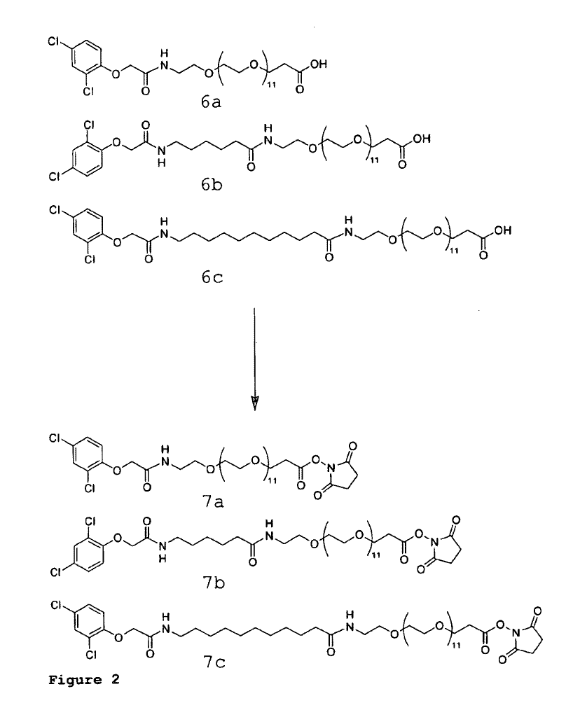

FIG. 2: Reaction scheme II.
Overview of the synthesis of some of the 2,4-dichlorophenoxyacetate derivatives of the kits of the invention containing oligoethyleneglycol moieties as described in the examples.

FIG. 3: 2,4-dichlorophenoxyacetate derivatives in peptide synthesis and their detection by an immuno-method (ELISA).
A 15mer epitope derived from ovalbumin (NH₂—(I)-GSIGAASMEFCFDCF-COOH, or NH₂-GSIGAAS-(II)-MEFCFDCF-COOH, or NH₂-GSIGAASMEFCFDCF-(III)-COOH) was SPOT-synthesized on a cellulose membrane. Epsilon-amino 2,4-D-labelled lysines (derivatives (2c), (4b), (4d)) bearing different spacers (-, C6 or C11) are inserted either amino- (I), or carboxyterminally (III) or within the 15mer sequence motif (II). Peptides terminally labelled with epsilon-amino 2,4-D-labelled lysine were equipped with biocytin at their respective opposite end. Peptides labelled within the sequence motif with epsilon-amino 2,4-D-labelled lysine were equipped with biocytin at the carboxyterminus (for details see example 10). Protection groups of the side chains were removed, peptides were cleaved-off from the membrane, lyophilized and dissolved in physiological buffer. Serially diluted, labelled peptide was captured on microplates that had been coated with monoclonal anti-2,4-D antibodies (clone E2/G2) and blocked with 1% (w/v) casein in phosphate buffered saline (Casein-PBS). Detection of the immobilized peptides was performed by applying streptavidin-horseradish peroxidase conjugate followed by 3,3′,5,5′-tetramethylbenzidine and hydrogen peroxide. 4-Parameter logistic functions were fitted onto the readouts and the EC₅₀-values (open bars) as well as the maximal optical densities (OD) at 450 nm (filled bars) were determined. *=Optical density significantly higher than that of constructs I, II, III- and IIIC6 (P<0.05; One-way ANOVA, Bonferroni Post hoc test).

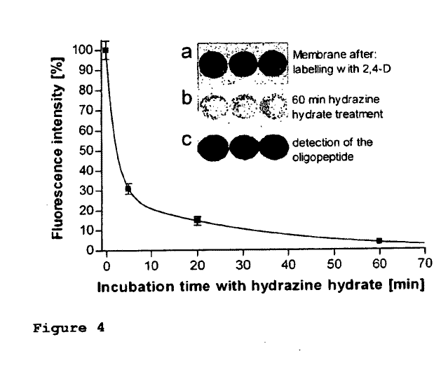

FIG. 4: Reversible labelling of peptides with 2-(2-(2,4-dichlorophenoxy)-1-hydroxyethylidene)-5,5-dimethylcyclohexane-1,3-dione (2,4-D-dimedone).
A 15mer epitope derived from ovalbumin (NH₂-GSIGAASMEFCFDCF-COOH) was SPOT-synthesized on cellulose and subsequently labelled aminoterminally with 2,4-D-dimedone (1c). After cleavage of the side chain protection groups the membrane was blocked with 1% (w/v) casein in phosphate buffered saline (Casein-PBS). The 2,4-D-label was visualized with a biotinylated anti-2,4-D antibody (clone E4/C2) and AlexaFluor680 labelled streptavidin and the fluorescence was quantitated (Inset a). For visualization of the cleavability the conjugate was treated with 5% (v/v) hydrazine hydrate in D-PBS and the remaining fluorescence was determined (Inset b). Integrity of the peptide upon removal of the label was confirmed by aminoterminal acetylation of the peptide, blocking in Casein-PBS, reaction with a monoclonal anti-ovalbumin antibody followed by AlexaFluor680 labelled secondary antibody (Inset c) and fluorescence quantitation.

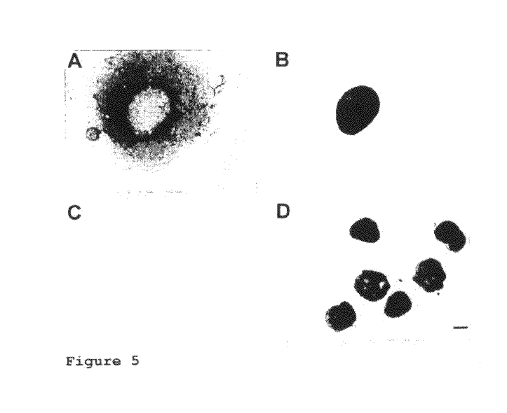

FIG. 5: Application of a 2,4-dichlorophenoxyacetic acid (2,4-D) labelled substrate molecule in immunohistochemistry.
A, B: Fixed and permeabilized human colon carcinoma (Caco-2) cells were blocked with 10% (v/v) fetal bovine serum (FBS) in Dulbecco's phosphate buffered saline (D-PBS) and subsequently incubated with 15 μM of wheat germ agglutinin which had been labelled with (3b) (2,4-D-WGA) in 5% (v/v) FBS in D-PBS. 2,4-D-WGA was detected with a 2,4-D specific monoclonal anti-body (clone E2/G2) and an anti-mouse IgG AlexaFluor546-labelled secondary antibody. 2,4-D-WGA bound predominantly to Golgi membranes (A). Cell nuclei were counterstained with 4′,6-diamidino-2-phenylindole dihydrochloride (DAPI) (B). C, D: Caco-2 cells were incubated following the same procedure, but omitting the monoclonal anti-2,4-D-antibody. The AlexaFluor546-labelled secondary antibody gave a negligible background signal (C), whereas cell nuclei could again be visualized with DAPI (D). The bar represents 10 μm.

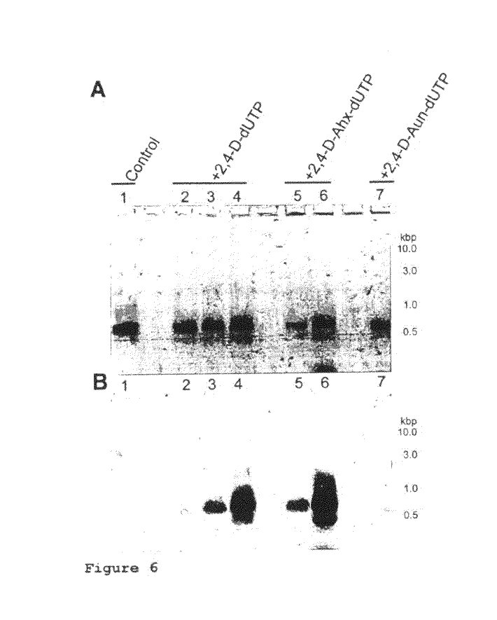

FIG. 6: Labelling of DNA-fragments with 2,4-dichlorophenoxyacetic acid-deoxyuridine-triphosphate derivatives (2,4-D-dUTP derivatives) via polymerase chain reaction (PCR).
A DNA-probe, a fragment (approx. 630 bp) from the murine prion protein gene, was amplified via PCR in absence or presence of various amounts of 2,4-D-dUTP derivatives containing no spacer (2,4-D-dUTP, (5a)), an aliphatic C6-spacer (2,4-D-Ahx-dUTP, (5b)), or an aliphatic C11-spacer (2,4-D-Aun-dUTP,(5c)) between the 2,4-D and the deoxyuridine-triphosphate (dUTP) moiety. A: 35 ng of the purified PCR-products were separated in a 1% (w/v) agarose gel and stained with ethidiumbromide. B: The separated DNA-probes were transferred to a nylon membrane and detected with an anti-2,4-D primary antibody (clone E4/C2) and an AlexaFluor680-labelled secondary antibody. Lane 1: control DNA-fragment which was amplified in presence of 0.4 nMoles unlabelled dUTP; lane 2-4: DNA-fragments which were amplified in the presence of increasing amounts (1, 5, or 10 μl) of (5a); lanes 5-6: DNA-fragments which were amplified in the presence of increasing amounts (1 or 5 μl) of (5b); lane 7: DNA-fragment which was amplified in the presence of 1 μl of (5c); bp: base pairs; kbp: kilo base pairs.

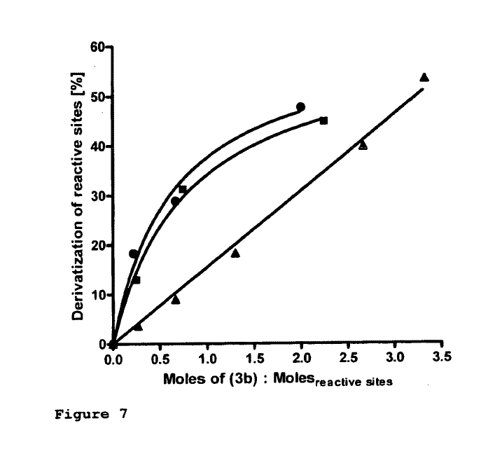

FIG. 7: Degree of 2,4-D-derivatization of proteinaceous substrate-molecules after labelling with (3b).
The substrate-molecules insulin (▴; 3 primary amino functions per molecule, equivalent to 3 reactive sites), ubiquitin (▪; 8 reactive sites) or recombinant Phl p 2-His6 (; 8 reactive sites) were derivatized with increasing amounts of (3b). The average degree of derivatization was determined by MALDI-TOF-MS analyses of the labelled conjugates and is given in percent derivatization of the reactive sites.

FIG. 8: 2,4-Dichlorophenoxyacetic acid-(2,4-D)-labelled nucleotide derivatives.
Overview of some 2,4-D-labelled nucleotide derivatives, which can be synthesized according to general description 5. (A) Nucleotide derivatives, which are 2,4-D-labelled at one of their phosphate moieties. (B) Nucleotide derivatives, which are 2,4-D-labelled at their carbohydrate moieties. (C) Nucleotide derivatives, which are 2,4-D-labelled at their purine or pyrimidine bases.

FIG. 9: 2,4-Dichlorophenoxyacetic acid-(2,4-D) derivatives containing oligoethyleneglycol-substructures.
Overview of some 2,4-dichlorophenoxyacetic acid-(2,4-D) derivatives containing oligoethyleneglycol-substructures, which can be synthesized according to general description 6.

### EXAMPLES

Examples 1 to 12 illustrate the syntheses of the compounds of the present invention, examples 13 to 20 illustrate their application as labelling reagents as well as the detection of labelled molecules by monoclonal 2,4-D-specific antibodies. FIGS. 1 and 2 give an overview of the syntheses illustrated in examples 1 to 12.

### General Description 1 for the Preparation of Alkylamino Acid Derivatives of 2,4-dichlorophenoxyacetic Acid

The respective alkylamino acid was dissolved at 37° C. in an equimolar amount of tetrabutylammoniumhydroxide solution (25% (w/v) in water). The solvent was removed in vacuo and the oily remainder was dried over phosphorous pentoxide (P₂O₅). 50 mMoles of the synthesized salt of the alkylaminoacid were dissolved along with 50 mMoles of (1b) in 200 ml anhydrous dioxane, 70 mMoles triethylamine were added and the solution was stirred over night at room temperature (RT). The solvent was removed in vacuo and the remainder was stirred with 40 ml ammonium hydroxide solution (resulting pH: 10). 250 ml 10% (w/v) citric acid were added (resulting pH: 5). The white precipitate was filtrated and washed with 50 ml 10% (w/v) citric acid and 40 ml water. The crude product was dissolved in dioxane and desalted with an excess of cation exchanger (Amberlite IR-120) for 30 minutes at RT. The resin was removed and the solvent evaporated in vacuo. The remainder was dried in a desiccator.

### General Description 2 for the Preparation of Succinimidylesters of 2,4-dichlorophenoxyacetic Acid (2,4-D) or 2,4-D-alkylamino Acid Derivatives

1 mMole of 2,4-D or of the respective alkylamino acid derivative of 2,4-dichlorophenoxyacetic acid was dissolved in 20 ml anhydrous dioxane. 1.1 mMoles pyridine were added. The reaction was started by addition of 2 mmoles of disuccinimidylcarbonate. The mixture was stirred for 48 h at RT. The solvent was removed in vacuo and the remainder was stirred with 20 ml of ice cold water for 60 min. The white solid product was filtrated, washed with ice cold water and dried in a desiccator over phosphorous pentoxide.

### General Description 3 for the Preparation of Sulfosuccinimidylesters of 2,4-dichlorophenoxyacetic Acid (2,4-D) or 2,4-D-alkylamino Acid Derivatives

1 mMole of 2,4-D or of the respective alkylamino acid derivative of 2,4-dichlorophenoxyacetic acid was dissolved in 3 ml of anhydrous N,N-dimethylformamide (DMF). An equimolar amount of N-hydroxysulfosuccinimide-sodium salt and 1.15 mMoles dicyclohexylcarbodiimide were added. The mixture was stirred over night at RT. The reaction was controlled by thin layer chromatography (silica gel 60; ethyl acetate; >95%). The mixture was cooled for 30 minutes on ice and the solid matter was filtered off. The solvent was removed in vacuo and the remainder was stirred with 25 ml ice cold ethanol for 40 min at RT. The white solid product was isolated, washed with ethanol and dried in a desiccator over phosphorous pentoxide.

### General Description 4 for the Preparation of N-alpha-(9-fluorenylmethyloxycarbonyl)-protected Amino Acid-Derivatives Labelled with 2,4-dichlorophenoxyacetic Acid (2,4-D) or 2,4-D-Derivatives at their Side Chains

0.3 mMoles of the respective activated carboxylic acid derivative of 2,4-dichlorophenoxyacetic acid, (1b), (3a) or (3c), were dissolved with an equimolar amount of N-alpha-(9-fluorenylmethyloxy-carbonyl)-L-lysine in 6 ml anhydrous N,N-dimethylformamide (DMF). 0.36 mMoles triethylamine were added. The solution was stirred over night at RT. Reaction progress was analyzed by thin layer chromatography (cyclohexane:aceton (1:1), 1% (v/v) trifluoroacetic acid). The crude products were isolated, applied onto a silica column (silica gel 60; cyclohexane:aceton (1:1), 1% (v/v) trifluoroacetic acid) and eluted. Fractions containing product were isolated and the solvents were evaporated. The product was stored dessiccated at 4° C.

In accordance with a modified general description 4 aspartic acid or glutamic acid derivatives which bear suitable protecting groups, such as N-alpha-Fmoc-aspartic acid alpha-t-butyl ester or N-alpha-Fmoc-glutamic acid alpha-t-butyl-ester, can be labelled with 2,4-D using derivative (1d). Tyrosine, serine or threonine derivatives bearing suitable protecting groups can be reacted according to a modified general description 4 by the application of alcohol reactive derivatives of 2,4-D according to formula (I). Such a derivative might be a halogen alkane which is reacted under basic conditions using an inert solvent such as tetrahydrofurane.

Cysteine derivatives which bear suitable protecting groups can be reacted with, for example, maleimide- or halogen alkane-modified 2,4-D labelling reagents in analogy to general description 4. Products can be purified using chromatographic techniques.

### General Description 5 for the Preparation of 2,4-dichlorophenoxyacetic Acid Labelled Nucleotide Derivatives

18 μMoles of the respective activated carboxylic acid derivative of 2,4-dichlorophenoxyacetic acid (1b, 3b or 3d) were dissolved in 360 μl anhydrous dimethylsulfoxide (DMSO). 8.5 μMoles of 5-(3-aminoallyl)-2′-deoxyuridine-5′-triphosphate (AA-dUTP) sodium salt were dissolved in 4.25 ml 100 mM sodium tetraborate buffer, pH 8.0. 750 μl thereof were added to 120 μl of the freshly prepared active ester solution of (1b), (3b) or (3d) in DMSO. The solutions were incubated on an end-to-end mixer for 4.5 hours at RT. The crude products were purified in several runs by anion exchange chromatography (MiniQ column on an AKTApurifier 10 HPLC, GE Healthcare, Uppsala, SWE) and were eluted with a linear gradient ranging from 20 mM to 1 M NH₄HCO₃ buffer (pH 7.9). Fractions containing product were isolated and the solvents were evaporated. The products were stored desiccated at −20° C.

In accordance with general description 5, nucleotides, such as cytidine, uridine, thymidine, adenosine or guanosine in their syn- or anti-configurations, each as mono-, di-, or triphosphate, or derivatives thereof, e.g. their cyclic forms, such as 3′-5′-cyclic adenosine monophosphate (cAMP), or their deoxy- or dideoxy-forms, or synthetic nucleoside analogues, such as xanthosine, hypoxanthosine, 6-mercaptopurine, 5-fluorouracil, 5-iodo-2′-deoxyuridine, 6-thioguanine, azothymidine or dideoxyinosine and their mono-, di-, and triphosphates (synthetic nucleotide analogues), can be labelled with 2,4-D. Prerequisite for the analogous synthesis according to general description 5 is the presence of at least one phosphate moiety and of a reactive group, such as a primary amine, an aldehyde or a thiol. Those reactive groups can be provided by modification of the purine or pyrimidine base, or of the carbohydrate moiety or of one of the phosphate moieties. Examples of such modified nucleotide derivatives, which form a particular aspect of the present invention, are N6-(4-amino)butyl-adenosine-5′-triphosphate, N6-(6-amino)hexyladenosine-5′-triphosphate, 8-[(4-amino)butyl]-amino-adenosine-5′-triphosphate, 8-[(6-amino)hexyl]-amino-adenosine-5′-triphosphate, 2′/3′-O-(2-aminoethyl-carbamoyl)-adenosine as 5′-di- or triphosphate, adenosine-5′-[-(4-aminophenyl)]triphosphate, gamma-[6-aminohexyl]-adenosine-5′-triphosphate, gamma-[(6-aminohexyl)-imido]-adenosine-5′-triphosphate, gamma-[(8-aminooctyl)-imido]-adenosine-5′-triphosphate, gamma-[6-aminohexyl]-guanosine-5′-triphosphate, gamma-[(6-aminohexyl)-imido]-guanosine-5′-triphosphate, gamma-[(8-aminooctyl)-imido]-guanosine-5′-triphosphate, 2′/3′-O-(2-aminoethyl-carbamoyl)-guanosine-5′-triphosphate, 8-[(6-amino)hexyl]-amino-guanosine-5′-monophosphate, 8-[(6-amino)hexyl]-amino-guanosine-3′,5′-cyclic monophosphate, 8-[(6-amino)hexyl]-amino-guanosine-5′-triphosphate, gamma-[(6-aminohexyl)-imido]-7-methyl-guanosine-5′-triphosphate, 2′/3′-O-(2-aminoethyl-carbamoyl)-7-methyl-guanosine-5′-di- or triphosphate, 5-(3-aminoallyl)-2′-deoxy-uridine-5′-triphosphate, gamma-[(6-aminohexyl)-imido]-2′-deoxy-uridine-5′-triphosphate, gamma-[(8-aminooctyl)-imido]-2′-deoxy-uridine-5′-triphosphate, 5-(3-aminoallyl)-uridine-5′-triphosphate, 5-(3-aminoallyl)-2′-deoxy-uridine-5′-[(alpha,beta)-methyleno]diphosphate, 5-(3-aminoallyl)-2′-deoxy-uridine-5′-[(alpha,beta)-methyleno]triphosphate, 5-propargylamino-2′-deoxy-cytidine-5′-triphosphate, or 5-propargylamino-cytidine-5′-triphosphate.

Analogous to general description 5, such nucleotide derivatives can be reacted with excess of reactive 2,4-D labelling derivatives. For example, a nucleotide equipped with a primary amino function can be reacted with labelling derivative (1b), (3b) or (3d), a nucleotide equipped with a carbonyl function with labelling derivative (1d), and a nucleotide equipped with a sulfhydryl group with a maleimide- or alkylhalogenide-derivative of 2,4-D. A purification of the products can be achieved by anion exchange chromatography. Dependent on their net charge, the products elute at different concentrations of salt solution, such as the above mentioned NH₄HCO₃-buffer. FIG. 8 gives an overview of possible 2,4-D labelled nucleotide derivatives.

### General Description 6 for the Preparation of 2,4-dichlorophenoxyacetic Acid Derivatives Containing Oligoethyleneglycol or Amino Acid Moieties

10 ml of a solution of 1.01 mmoles (850 mg) Fmoc-NH-PEG₁₁-COOH in anhydrous dichloromethane (DCM) were mixed with 3.80 mMoles (650 μl) diisopropylethylamine (DIPEA), dried for 15 min with molecular sieves (3 Å) and subsequently added to 1 g 2-chlorotritylchloride-resin (load: 1.1 mMoles/g). After 120 min under an N₂ atmosphere the resin was washed 3× with 15 ml of DCM/methanol/DIPEA (17:2:1) and 3× with 15 ml of DCM each and was subsequently dried over night over sodium hydroxide. The 2-chlorotrityl-(Fmoc-NH-PEG₁₁) resin was divided into 3 portions of 500 mg each which were used for the synthesis of the derivatives (6a), (6b) and (6c) respectively. For synthesis of derivatives (6b) and (5c) two portions of the 2-chlorotrityl-(Fmoc-NH-PEG₁₁) resin were swollen for 30 min in 10 ml DCM each, treated 2× for 10 min with 10 ml 20% (v/v) piperidine/N,N-dimethylformamide (DMF) and washed 5× with 10 ml DMF. 3.0 mMoles of the respective Fmoc-aminoalkanoic acid and 3.0 mmoles (1.241 g) of O-(1H-6-chlorobenzotriazole-1-yl)-1,1,3,3-tetramethyluronium-hexafluorophosphate (HCTU) were dissolved in 5 ml of DMF, 5.85 mmoles (1 ml) DIPEA were added and the resulting solutions were added to the respective portions of deprotected resin. After 120 min under inert gas (N₂) atmosphere the resin portions were washed 3× with 10 ml DMF, treated 2× for 10 min with 10 ml 20% (v/v) of piperidin/DMF and washed 5× with 10 ml DMF. The two 2-chlorotrityl-(Fmoc-aminoalkanoic acid-amide-PEG₁₁) resin portions were converted into derivatives (6b) and (6c) in a second synthesis step. For generation of derivative (6a) the third portion of the 2-chlorotrityl-(Fmoc-NH-PEG₁₁) resin which had not been derivatized further with an aminoalkanoic acid, was used in this step. 3.0 mMoles (663 mg) of 2,4-dichlorphenoxyacetic acid and 3.0 mMoles (1.241 g) of HCTU were dissolved in 5 ml of DMF, 5.85 mmoles (1 ml) of DIPEA was added and the resulting solutions were added to the respective resin samples. After 120 min under inert gas (N₂) atmosphere the resin samples were washed 3× with 10 ml DMF and 3× with 10 ml DCM each. The products were cleaved 2× for 10 min from the resin with 15 ml of a solution of 10% (v/v) trifluoroacetic acid (TFA) in DCM and the resins were finally washed 3× with 10 ml DCM each. These solutions were combined and the solvents evaporated. The remainders were taken up in 2 ml water each and were centrifugated for 5 min at 16000 g. The supernatants were isolated and lyophilized. The oily residues were used for the preparations of the NHS-esters (7a), (7b) and (7c) without further purification.

In accordance with general description 6, other 2,4-D derivatives containing oligoethyleneglycol moieties can be synthesized. Such oligoethyleneglycol moieties are for example linear or branched ethyleneglycol homopolymers, or propyleneglycol homopolymers, as well as mixed ethyleneglycol/propyleneglycol copolymers with average molecular weights of 100 to 5000 g/Mole which can be substituted on one or more sites. Explicitly included are 1-ethoxy-2-ethoxy-ethyl, 1-ethoxy-2-(2-ethoxyethoxy)ethyl, 1-ethoxy-2-[2-(2-ethoxyethoxy)ethoxy]ethyl, and homologues thereof.

Prerequisite for the analogous reaction according to general description 6, the starting component containing the respective oligoethyleneglycol moiety must be equipped with a fluorenylmethoxycarbonyl- (Fmoc) or a (1,1,-dioxobenzo[b]thiophene-2-yl-methyl)oxycarbonyl- (Bsmoc) protected aminofunction, as well as with a free carboxy-, hydroxyl-, amino-, imidazol-, phenol-, or thiolfunction. Examples for such a component are Fmoc-NH-PEG₂-COOH (MW 559), Fmoc-NH-PEG₁₁-propionic acid (MW 840), or Fmoc-NH-PEG₂₇-propionic acid (MW 1545).

Analogous to general description 6 such an oligoethyleneglycol-containing component can be coupled in the first synthesis step to the 2-chlorotritylchloride resin. After deprotection the resin can be reacted either directly with 2,4-D or, in a first step, with an Fmoc- or Bsmoc-aminoalkanoic acid followed by reaction with 2,4-D in a second step, as described above. Finally, the product is cleaved off the resin. FIG. 9 shows a selection of 2,4-D derivatives containing oligoethyleneglycol moieties.

In accordance with general description 6, other 2,4-D derivatives containing amino acids can be synthesized. As a prerequisite, the amino acid derivatives must be N-terminally protected with Fmoc- or Bsmoc-protection groups and, if applicable, with orthogonal protection groups at their side chains. Orthogonal protection groups can be e.g. S-acetamidomethyl- (Acm), t-butyloxycarbonyl- (Boc), t-butyl (tBu), trityl- (Trt), 2,2,4,6,7-pentamethyldihydrobenzofurane-5-sulfonyl (Pbf), tosyl- (Ts), benzyloxycarbonyl (CBz) or beta-2-adamantyl (Ada). Possible amino acid derivatives comprise all natural amino acids, preferably alanine, beta-alanine, aspartic acid, asparagine, arginine, citrulline, cysteine, glycine, glutamic acid, glutamine, histidine, homoserine, hydroxyproline, isoleucine, leucine, lysine, methionine, ornithine, phenylalanine, proline, sarcosine, serine, threonine, tryptophane, tyrosine and valine, each in L- and D-configuration, as well as non-natural amino acids, e.g. 2-aminoethylglycine, phenylglycine, penicillamine, norvaline, norleucine, alpha-aminobutyric acid, diaminopropionic acid, cyclohexylalanine, butylglycine, aminoisobutyric acid, thienylalanine, statine, each in L- and D-configuration, and aminooligoethyleneglycol-carboxylic acids and aminooligopropyleneglycol-carboxylic acids. Examples for such protected amino acid derivatives are Fmoc-alanine-OH, Fmoc-arginine(Pbf)-OH, Fmoc-asparagine(Trt)-OH, Fmoc-aspartic acid (tBu)-OH, Fmoc-cysteine(Trt)-OH, Fmoc-glutamine(Trt)-OH, Fmoc-glutamic acid(tBu)-OH, Fmoc-glycine-OH, Fmoc-histidine(Trt)-OH, Fmoc-isoleucine-OH, Fmoc-leucine-OH, Fmoc-lysine(Boc)-OH, Fmoc-methionine-OH, Fmoc-phenylalanine-OH, Fmoc-proline-OH, Fmoc-serine (tBu)-OH, Fmoc-threonine (tBu)-OH, Fmoc-tryptophane(Boc)-OH, Fmoc-tyrosine(tBu)-OH, or Fmoc-valine-OH.

In accordance with general description 6 the respective amino acid derivatives can be coupled in the first synthesis step to the 2-chlorotritylchloride resin. After deprotection the resin can be reacted either directly with 2,4-D or, in a first step with Fmoc- or Bsmoc-aminoalkanoic acid followed by reaction with 2,4-D in a second step as described above. Finally, the product is cleaved from the resin using TFA in a concentration of up to 10% (v/v).

### Example 1

### Preparation of 2,4-dichlorophenoxyacetic acid-N-hydroxysuccinimidyl-ester (1b)

0.08 Moles 2,4-dichlorophenoxyacetic acid (17.6 g) and 0.08 Moles N-hydroxysuccinimide (9.2 g) were dissolved in 180 ml anhydrous dioxane. An equimolar amount of dicyclohexylcarbodiimide (0.08 Moles, 16.5 g) was added. The mixture was stirred over night at RT. The solid matter was filtered off and washed with dioxane. Filtrate and dioxane wash were pooled and evaporated, and the residue was washed with 60 ml methanol:ethanol (1:1) followed by diethylether. The isolated white solid product was dried in a desiccator over phosphorous pentoxide. Yield: 73% (R_{f} (silica gel; ethyl acetate)=0.86).

¹H-NMR (360 MHz, CDCl₃) δ2.86 (s, 4H), 5.02 (s, 2H), 6.91 (d, 1H), 7.22 (dd, 1H), 7.40 (d, 1H).

¹³C-NMR (90.6 MHz, CDCl₃) δ25.6, 64.5, 115.5, 124.6, 127.8, 128.1, 130.5, 151.8, 164.0, 168.5.

### Example 2

### Preparation of 6-(2-(2,4-dichlorophenoxy)acetylamido)-hexyl amine (1d)

1 mMole of (1b) (301 mg) was incubated over night at RT with 1 mMole (253 mg) N-Boc-1,6-diaminohexanehydrochloride and 1.2 mmole (206 μl) diisopropylethylamine (DIPEA) in 15 ml anhydrous dioxane/N,N-dimethylformamide (DMF) (1:2). The reaction progress was controlled by thin layer chromatography (toluene:acetone (2:1), R_{f}=0.44). After 22 h the solvent was removed in vacuo. The crude product was stirred for 30 min at RT with 30 ml ice-cold water and was isolated by filtration. The product was desiccated and stored dry at RT (Yield 78%). The N-Boc-protection group was removed in 7 ml dioxane/trifluoroacetic acid (TFA) (1:1) for 180 min at RT. The progress of deprotection was controlled by thin layer chromatography. Another 3.5 ml of TFA were added and the solution was further stirred for 210 min at RT. After complete deprotection of (1d) the solvent was removed in vacuo. The crude product was washed with dioxane and ice-cold toluene and was isolated by filtration. Residual solvent was evaporated and (1d) was stored desiccated at 4° C. Yield over two steps 59%.

¹H-NMR (360 MHz, DMSO) δ 1.27 (m, 4H), 1.42 (m, 2H), 1.51 (m, 2H), 3.12 (q, 2H), 4.60 (s, 2H), 7.04 (d, 1H), 7.36 (dd, 1H), 7.59 (d, 1H).

¹³C-NMR (90.6 MHz, DMSO) δ 25.3, 25.7, 26.7, 26.8, 28.9, 38.0, 38.1, 67.9, 115.3, 122.4, 125.0, 127.9, 129.3, 152.4, 166.6.

MS-ESI (m/z): 318.091 [M] (calculated for C₁₄Cl₂H₂₀N₂O₂: 318.091).

### Example 3

### Preparation of 6-(2-(2,4-dichlorophenoxy)acetylamino)-hexanoic Acid (2a)

0.05 Moles (18.67 g) of 6-aminohexanoic acid-tetrabutylammonium salt were reacted with 0.05 Moles (15.05 g) (1b) in 200 ml anhydrous dioxane and 0.07 Moles (10 ml) triethylamine according to general description 1. The yield was 90%. The crude product was recrystallized from toluene (purity (RP-HPLC, Jupiter C18)>98%).

¹H-NMR (360 MHz, CDCl₃) δ1.41 (m, 2H), 1.60 (m, 2H), 1.68 (m, 2H), 2.36 (t, 2H), 3.38 (q, 2H), 4.52 (s, 2H), 6.84 (d, 1H), 7.23 (dd, 1H), 7.41 (d, 1H).

¹³C-NMR (90.6 MHz, CDCl₃) δ24.2, 26.2, 29.1, 33.7, 38.9, 68.3, 114.7, 123.7, 127.5, 128.1, 130.2, 151.6, 167.3, 178.4.

MS-ESI m/z: 333.053 [M] (calculated for C₁₄Cl₂H₁₇NO₄: 333.053).

### Preparation of 11-(2-(2,4-dichlorophenoxy)acetylamino)-undecanoic Acid (2b)

0.05 Moles (22.17 g) of 11-aminoundecanoic acid-tetrabutylammonium salt were reacted with 0.05 Moles (1b) (6.34 g) in 200 ml anhydrous dioxane and 0.07 Moles (10 ml) triethylamine according to general description 1. The yield was 81%. The crude product was dissolved in toluene:acetone (1:1) applied on a silica gel column and eluted (purity (RP-HPLC, Jupiter C18)>98%).

¹H-NMR (360 MHz, CDCl₃) δ1.31 (m, 12H), 1.56 (m, 2H), 1.63 (m, 2H), 2.34 (t, 2H), 3.36 (q, 2H), 4.52 (s, 2H), 6.84 (d, 1H), 7.22 (dd, 1H), 7.41 (d, 1H).

¹³C-NMR (90.6 MHz, CDCl₃) δ24.7, 26.7, 29.0, 29.1, 29.2, 29.3, 33.9, 39.1, 68.3, 114.7, 123.7, 127.4, 128.0, 130.2, 151.6, 167.2, 178.8.

MS-ESI m/z: 403.130 [M] (calculated for C₁₉Cl₂H₂₇NO₄: 403.132).

### Example 4

### Preparation of 6-(2-(2,4-dichlorophenoxy)acetylamino)-hexanoic acid-N-hydroxysuccinimidyl-ester (3a)

1 mMole (0.33 g) of 6-(2-(2,4-dichlorophenoxy)acetylamino)hexanoic acid was reacted with 2 mMoles (0.51 g) disuccinimidyl-carbonate in 20 ml anhydrous dioxane and 1.1 mMoles (82 μl) pyridine according to general description 2. The yield was 90%.

¹H-NMR (360 MHz, CDCl₃) δ1.48 (m, 2H), 1.62 (m, 2H), 1.79 (m, 2H), 2.62 (t, 2H), 2.81 (s, 4H), 3.39 (q, 2H), 4.51 (s, 2H), 6.85 (d, 1H), 7.24 (dd, 1H), 7.41 (d, 1H).

¹³C-NMR (90.6 MHz, CDCl₃) δ24.2, 25.6, 25.9, 28.9, 30.8, 38.8, 68.3, 114.7, 123.8, 127.4, 128.0, 130.2, 151.7, 167.1, 168.4, 169.1.

MS-ESI m/z: 430,070 [M] (calculated for C₁₈Cl₂H₂₀N₂O₆: 430.070).

### Preparation of 11-(2-(2,4-dichlorophenoxy)acetylamino)-undecanoic acid-N-hydroxysuccinimidyl-ester (3c)

1 mMole (0.40 g) of 11-(2-(2,4-dichlorophenoxy)acetylamino)undecanoic acid was reacted with 2 mMoles (0.51 g) disuccinimidyl-carbonate in 20 ml anhydrous dioxane and 1.1 mMoles (82 μl) pyridine according to general description 2. Yield 90%.

¹H-NMR (360 MHz, CDCl₃) δ1.29 (m, 10H), 1.40 (m, 2H), 1.56 (m, 2H), 1.74 (m, 2H), 2.60 (t, 2H), 2.83 (s, 4H), 3.36 (q, 2H), 4.51 (s, 2H), 6.84 (d, 1H), 7.22 (dd, 1H), 7.41 (d, 1H).

¹³C-NMR (90.6 MHz, CDCl₃) δ24.5, 25.6, 26.8, 28.7, 29.0, 29.1, 29.2, 29.3, 29.4, 30.9, 39.1, 68.3, 114.6, 123.7, 127.4, 128.0, 130.2, 151.6, 167.0, 168.6, 169.1.

MS-ESI m/z: 500.147 [M] (calculated for C₂₃Cl₂H₃₀N₂O₆: 500.148).

### Example 5

### Preparation of 2-(2,4-dichlorophenoxy)acetic acid-hydrazide (1a)

50 mMoles (11 g) of 2,4-dichlorophenoxyacetic acid were refluxed with a 25 fold molar excess of thionylchloride (90 ml) for 2 h. Excess thionylchloride was removed by destillation. 2,4-dichlorophenoxyacetylchloride was dissolved in 50 ml anhydrous dioxane and stirred with an equimolar amount of hydrazine monohydrate over night at RT. The solution was evaporated. Yield >90%.

¹H-NMR (360 MHz, DMSO/CDCl₃ (1:1)) δ4.60 (d, 2H), 7.01 (d, 1H), 7.23 (dd, 1H), 7.39 (d, 1H).

¹³C-NMR (90.6 MHz, DMSO/CDCl₃ (1:1)) δ66.2, 113.8, 121.9, 124.7, 126.3, 128.1, 151.0, 165.0.

MS-ESI m/z: 233.996 [M] (calculated for C₈Cl₂H₈N₂O₂: 233.996).

### Preparation of 6-(2-(2,4-dichlorophenoxy)acetylamino)-hexanoic acid-hydrazide (4a)

0.5 mMoles (215 mg) of (3a) were dissolved in 10 ml anhydrous dioxane. The solution was dropped slowly to a solution of hydrazine monohydrate (5 mMoles) in dioxane (60 ml). The mixture was stirred over night at RT. Precipitate was filtered off and discarded. The filtrate was evaporated and the remainder was stirred for 10 min with 30 ml ice cold water. The white precipitate was isolated and desiccated over phosphorous pentoxide. Yield 66%.

¹H-NMR (360 MHz, CDCl₃) δ1.36 (m, 2H), 1.57 (m, 2H), 1.67 (m, 2H), 2.24, 2.55 (t, 2H), 3.35 (m, 2H), 4.51 (d, 2H), 6.86 (m, 1H), 7.21 (m, 1H), 7.38, 7.41 (d, 1H).

¹³C-NMR (90.6 MHz, CDCl₃) δ24.7, 24.9, 26.0, 26.2, 29.0, 29.1, 33.9, 38.8, 67.1, 68.3, 114.7, 114.9, 123.7, 127.4, 128.1, 130.1, 130.2, 151.6, 167.5, 173.3.

MS-ESI m/z: 347.079 [M] (calculated for C₁₄Cl₂H₁₉N₃O₃: 347.080).

### Preparation of 11-(2-(2,4-dichlorophenoxy)acetylamino)-undecanoic acid-hydrazide (4c)

0.5 mMoles (250 mg) of (3c) were dissolved in 10 ml anhydrous dioxane. The solution was dropped slowly to a solution of hydrazine monohydrate (5 mMoles) in dioxane (60 ml). The mixture was stirred over night at RT. The precipitated, white product was isolated, washed with water and desiccated over phosphorous pentoxide. Yield 90%.

¹H-NMR (360 MHz, DMSO) δ1.24 (m, 12H), 1.43 (m, 2H), 1.49 (m, 2H), 2.01, 2.20 (t, 2H), 3.13 (q, 2H), 4.61 (s, 2H), 7.06 (d, 1H), 7.37 (dd, 1H), 7.60 (d, 1H).

¹³C-NMR (90.6 MHz, CDCl₃) δ24.4, 25.1, 26.2, 28.4, 28.5, 28.6, 28.7, 28.8, 28.9, 33.3, 33.6, 38.1, 38.2, 66.2, 67.9, 115.3, 122.4, 125.0, 127.9, 129.2, 152.4, 166.4, 166.5, 171.5.

MS-ESI m/z: 417.157 [M] (calculated for C₁₉Cl₂H₂₉N₃O₃: 417.159).

### Example 6

### Preparation of 6-(2-(2,4-dichlorophenoxy)acetylamino)-hexanoic acid-N-hydroxysulfosuccinimidyl-ester-sodium Salt (3b)

1 mMoles (333 mg) of (2a) was reacted with 1 mMole (218 mg) N-hydroxysulfosuccinimide-sodium salt and 1.15 mMoles (237 mg) dicyclohexylcarbodiimide in 3 ml anhydrous N,N-dimethylformamide (DMF) according to general description 3. Yield 53%.

¹H-NMR (360 MHz, DMSO) δ1.34 (m, 2H), 1.46 (m, 2H), 1.62 (m, 2H), 2.64 (t, 2H), 2.86 (dd, 1H), 3.12 (q, 2H), 3.16 (m, 1H), 3.94 (m, 1H), 4.60 (s, 2H), 7.04 (d, 1H), 7.36 (dd, 1H), 7.58 (d, 1H).

¹³C-NMR (90.6 MHz, DMSO) δ23.8, 25.2, 28.3, 30.0, 30.8, 37.8, 38.0, 56.2, 67.8, 115.3, 122.5, 124.9, 127.9, 129.2, 152.5, 165.3, 166.5, 166.6, 168.7.

MS-ESI m/z: 510.026 [M] (calculated for C₁₈Cl₂H₂₀N₂O₉S: 510.027).

### Preparation of 11-(2-(2,4-dichlorophenoxy)acetylamino)-undecanoic acid-N-hydroxysulfosuccinimidyl-ester-sodium Salt (3d)

1 mMole (404 mg) of (2b) was reacted with 1 mMole (218 mg) N-hydroxysulfosuccinimide-sodium salt and 1.15 mMoles (237 mg) dicyclohexylcarbodiimide in 3 ml anhydrous N,N-dimethylformamide (DMF) according to general description 3. Yield 76%.

¹H-NMR (360 MHz, DMSO) δ1.24 (m, 10H), 1.34 (m, 2H), 1.41 (m, 2H), 1.61 (m, 2H), 2.63 (t, 2H), 2.85 (dd, 1H), 3.11 (q, 2H), 3.15 (m, 1H), 3.93 (m, 1H), 4.60 (s, 2H), 7.04 (d, 1H), 7.35 (dd, 1H), 7.58 (d, 1H).

¹³C-NMR (90.6 MHz, DMSO) δ24.2, 26.2, 27.9, 28.4, 28.6, 28.7, 28.8, 30.1, 30.8, 38.1, 56.2, 67.8, 115.3, 122.4, 124.9, 127.9, 129.2, 152.4, 165.2, 166.4, 166.5, 168.7.

MS-ESI m/z: 580.103 [M] (calculated for C₂₃Cl₂H₃₀N₂O₉S: 580.105).

### Example 7

### Preparation of 6-(2-(2,4-dichlorophenoxy)acetylamino)-2-(9H-fluoren-9-yl-methoxycarbonylamino)-hexanoic acid (2c)

0.3 mMoles (100 mg) of (1b) were reacted with 0.3 mMoles (110 mg) N-alpha-(9-fluorenylmethyloxycarbonyl)-L-lysine in 6 ml anhydrous N,N-dimethylformamide (DMF) and 0.36 mMoles (50 μl) triethylamine according to general description 4. Yield 76%.

¹H-NMR (360 MHz, CDCl₃) δ1.35 (m, 2H), 1.45 (m, 2H), 1.57 (m, 2H), 3.12 (q, 2H), 3.91 (m, 1H), 4.25 (m, 3H), 4.58 (m, 2H), 7.05 (m, 1H), 7.36 (m, 4H), 7.66 (m, 2H), 7.88 (m, 5H), 7.95 (m, 1H).

¹³C-NMR (90.6 MHz, CDCl₃) δ22.9, 28.4, 30.3, 38.1, 46.6, 53.6, 65.5, 67.8, 115.3, 120.0, 122.5, 125.0, 125.1, 125.2, 126.9, 127.5, 127.9, 128.5, 129.2, 131.4, 140.6, 143.7, 152.4, 156.1, 166.5, 166.8, 172.6, 173.8.

MS-ESI m/z: 570.131 [M] (calculated for C₂₉Cl₂H₂₈N₂O₆: 570.132).

### Preparation of 6-(6-(2-(2,4-dichlorophenoxy)acetylamino)-hexanoylamino)-2-(9H-fluoren-9-yl-methoxycarbonylamino)-hexanoic Acid (4b)

0.3 mMoles (130 mg) of (3a) were reacted with 0.3 mMoles (110 mg) N-alpha-(9-fluorenylmethyloxycarbonyl)-L-lysine in 6 ml anhydrous N,N-dimethylformamide (DMF) and 0.36 mMoles (50 μl) triethylamine according to general description 4. Yield 72%.

¹H-NMR (360 MHz, CDCl₃) δ1.40 (m, 8H), 1.65 (m, 2H), 2.03 (m, 4H), 3.11 (m, 4H), 3.89 (m, 1H), 4.23 (m, 3H), 4.59 (s, 2H), 7.05 (d, 1H), 7.32 (m, 3H), 7.41 (t, 2H), 7.53 (d, 1H), 7.75 (d, 2H), 7.85 (d, 2H).

¹³C-NMR (90.6 MHz, CDCl₃) δ23.1, 25.0, 26.0, 28.7, 28.8, 29.5, 30.5, 35.3, 35.7, 38.2, 38.3, 46.7, 53.8, 65.6, 67.9, 113.0, 115.4, 120.0, 122.6, 125.1, 125.2, 125.3, 127.0, 127.6, 128.0, 129.3, 140.7, 143.8, 152.5, 156.2, 162.3, 166.6, 171.9, 173.9.

MS-ESI m/z: 683.202 [M] (calculated for C₃₅Cl₂H₃₉N₃O₇: 683.217).

### Preparation of 6-(11-(2-(2,4-dichlorophenoxy)acetylamino)-undecanoylamino)-2-(9H-fluoren-9-yl-methoxycarbonylamino)-hexanoic Acid (4d)

0.3 mMoles (150 mg) of (3c) were reacted with 0.3 mMoles (110 mg) N-alpha-(9-fluorenylmethyloxycarbonyl)-L-lysine in 6 ml anhydrous N,N-dimethylformamide (DMF) and 0.36 mMoles (50 μl) triethylamine according to general description 4. Yield 61%.

¹H-NMR (360 MHz, CDCl₃) δ1.18 (m, 12H), 1.41 (m, 8H), 1.62 (m, 2H), 2.02 (t, 2H), 3.02 (m, 2H), 3.15 (m, 2H), 3.87 (m, 1H), 4.26 (m, 3H), 4.58 (s, 2H), 7.05 (d, 1H), 7.32 (m, 3H), 7.41 (t, 2H), 7.58 (d, 1H), 7.71 (d, 2H), 7.87 (d, 2H).

¹³C-NMR (90.6 MHz, CDCl₃) δ23.1, 25.2, 25.3, 26.3, 28.7, 28.8, 28.9, 29.0, 30.4, 30.7, 35.4, 35.5, 38.0, 38.1, 38.3, 46.7, 53.8, 65.6, 68.0, 115.4, 120.1, 122.6, 125.1, 125.3, 125.3, 127.1, 127.6, 128.0, 140.7, 143.8, 143.9, 152.5, 156.2, 157.9, 158.2, 166.5, 166.6, 171.9, 172.0, 172.7, 173.9.

MS-ESI m/z: 753.301 [M] (calculated for C₄₀Cl₂H₄₉N₃O₇: 753.295).

### Example 8

### Preparation of 2-(2-(2,4-dichlorophenoxy)-1-hydroxyethylidene)-5,5-dimethylcyclohexane-1,3-dione (1c)

15 mMoles (3.3 g) 2,4-dichlorophenoxyacetic acid were dissolved in 150 ml anhydrous dichloromethane. 16.5 mMoles (2.3 g) 5,5-dimethyl-1,3-cyclohexanedione (dimedone), 15 mMoles (3.1 g) dicyclohexylcarbodiimide and 15 mMoles (1.8 g) 4-dimethylaminopyridine were added. The mixture was stirred 48 h at RT. The synthesis residue was filtered off and the filtrate was evaporated. The remainder was extracted with 50 ml ethyl acetate and filtered. The filtrate was washed with 1 M potassium hydrogensulfate solution and subsequently with 1 M sodium hydrogencarbonate solution. The organic phase was isolated and evaporated. The remainder was washed with diethyl ether. The solid product was isolated and desiccated over phosphorous pentoxide. Yield 19% (Mp. 170-175° C., R_{f} (silica gel, ethyl acetate)=0.8). An additional extraction of the synthesis residue increased the yield to 24-29%.

¹H-NMR (360 MHz, DMSO) δ0.95 (s, 6H), 2.11 (s, 4H), 5.06 (s, 2H), 6.69 (d, 1H), 7.24 (d, 1H), 7.47 (d, 1H).

¹³C-NMR (90.6 MHz, DMSO) δ28.2, 29.8, 52.7, 74.2, 112.4, 114.9, 121.6, 123.2, 127.6, 128.7, 153.6, 189.3, 194.2.

MS-ESI m/z: 342.042 [M] (calculated for C₁₆Cl₂H₁₆O₄: 342.043).

### Example 9

### Preparation of 2,4-dichlorophenoxyacetic acid-[5-(3-amidoallyl)-2′-deoxyuridine-5′-triphosphate] (5a)

6 μMoles (1.8 mg) of (1b) were reacted with 1.5 μMoles (0.9 mg) 5-(3-aminoallyl)-2′-deoxyuridine-5′-triphosphate sodium salt in 870 μl 100 mM sodium tetraborate buffer, pH 8.0, containing 14% (v/v) anhydrous dimethylsulfoxide according to general description 5. The product (5a) eluted from the anion exchange column between 54.5±2.9% and 71.8±2.6% of 1 M NH₄HCO₃ buffer, pH 7.9.

¹H-NMR (360 MHz, D₂O) δ 2.35 (m, 2H), 3.93 (m, 2H), 4.17 (m, 3H), 4.61 (m, 2H), 6.18 (m, 1H), 6.25 (m, 1H), 6.33 (m, 1H), 6.95 (m, 1H), 7.23 (m, 1H), 7.46 (m, 1H), 7.81 (s, 1H).

MS-ESI m/z: 724.970 [M] (calculated for C₂₀Cl₂H₂₄N₃O₁₆P₃: 724.975).

### Preparation of 6-(2-(2,4-dichlorophenoxy)acetylamido)-hexanoic acid-[5-(3-amidoallyl)-2′-deoxyuridine-5′-triphosphate] (5b)

6 μMoles (3.2 mg) of (3b) were reacted with 1.5 μMoles (0.9 mg) 5-(3-aminoallyl)-2′-deoxyuridine-5′-triphosphate sodium salt in 870 μl 100 mM sodium tetraborate buffer, pH 8.0, containing 14% (v/v) anhydrous dimethylsulfoxide according to general description 5. The product (5b) eluted from the anion exchange column between 46.0±2.5% and 65.5±3.0% of 1 M NH₄HCO₃ buffer, pH 7.9.

¹H-NMR (360 MHz, D₂O) δ 1.26 (m, 2H), 1.42 (m, 2H), 1.51 (m, 2H), 1.59 (m, 2H), 2.25 (m, 4H), 3.27 (m, 2H), 3.84 (m, 2H), 4.13 (m, 3H), 4.55 (m, 2H), 6.08 (m, 2H), 6.21 (m, 1H), 6.79 (m, 1H), 7.18 (m, 1H), 7.85 (m, 1H), 7.68 (s, 1H).

MS-ESI m/z: 837.050 [M] (calculated for C₂₆Cl₂H₃₄N₄O₁₇P₃: 837.050).

### Preparation of 11-(2-(2,4-dichlorophenoxy)acetylamido)-undecanoic acid-[5-(3-amidoallyl)-2′-deoxyuridine-5′-triphosphate] (5c)

3 μMoles (1.8 mg) of (3d) were reacted with 0.75 μMoles (0.45 mg) 5-(3-aminoallyl)-2′-deoxyuridine-5′-triphosphate sodium salt (AA-dUTP) according to general description 5. In variation to general description 5 3 μMoles of (3d) dissolved in 60 μl anhydrous dimethylsulfoxide (DMSO) were added to a solution of 0.75 μMoles AA-dUTP in 375 μl 100 mM sodium tetraborate buffer, pH 8.0, 2.24 ml water and 367 μl anhydrous DMSO. After 4.5 h incubation at RT approximately 95% of the solvent were removed in vacuo and the remainder was dissolved in 300 μl water for further purification by anion exchange chromatography. The product (5c) eluted from the anion exchange column between 71.0±2.6% and 97.9±2.8% of 1 M NH₄HCO₃ buffer, pH 7.9.

MS-ESI m/z: 908.134 [M] (calculated for C₃₁Cl₂H₄₅N₄O₁₇P₃: 908.137).

### Example 10

### Preparation of 6-(2-(2,4-dichlorophenoxy)acetylamido)-PEG₁₁-carboxylic Acid (6a)

500 mg of 2-chlorotrityl-(Fmoc-NH-PEG₁₁) resin were reacted with 3.0 mMoles (663 mg) 2,4-dichlorophenoxyacetic acid according to general description 6. A yellow oily product was isolated. Yield 58%.

¹H-NMR (360 MHz, CDCl₃) δ 2.61 (t, 2H), 3.57 (t, 2H), 3.64 (m, 46H), 3.78 (t, 2H), 4.55 (s, 2H), 6.86 (d, 1H), 7.22 (dd, 1H), 7.41 (d, 1H).

¹³C-NMR (90.6 MHz, CDCl₃) δ 34.9, 38.9, 66.6, 68.3, 69.5, 70.2, 70.4, 70.5, 70.6, 70.7, 77.2, 114.8, 123.8, 127.4, 128.0, 130.2, 151.7, 167.5, 173.4.

MS-ESI m/z: 933.305 [M+TFA] (calculated for C₃₅Cl₂H₅₉NO₁₆: 819.318).

### Preparation of (6-(2-(2,4-dichlorophenoxy)acetylamido) hexylamido)-PEG₁₁-carboxylic Acid (6b)

500 mg of 2-chlorotrityl-(Fmoc-NH-PEG₁₁) resin were reacted with 3.0 mMoles (1.06 g) Fmoc-aminohexanoic acid in the first synthesis step and 3.0 mMoles (663 mg) 2,4-dichlorophenoxy-acetic acid in the second step according to general description 6. A yellow oily product was isolated. Yield 58%.

¹H-NMR (360 MHz, CDCl₃) δ 1.39 (m, 2H), 1.60 (quin, 2H), 1.69 (quin, 2H), 2.29 (t, 2H), 2.61 (t, 2H), 3.37 (q, 2H), 3.46 (m, 2H), 3.58 (t, 2H), 3.65 (m, 44H), 3.78 (t, 2H), 4.54 (s, 2H), 6.86 (d, 1H), 7.23 (dd, 1H), 7.41 (d, 1H).

¹³C-NMR (90.6 MHz, CDCl₃) δ 25.3, 26.3, 29.0, 34.9, 35.8, 39.0, 39.7, 66.6, 68.2, 69.5, 70.2, 70.3, 70.4, 70.5, 70.6, 77.0, 114.9, 123.8, 127.5, 128.1, 130.2, 151.6, 167.7, 173.4, 174.4.

MS-ESI m/z: 932.404 [M] (calculated for C₄₁Cl₂H₇₀N₂O₁₇: 932.405).

### Preparation of (6-(2-(2,4-dichlorophenoxy)acetylamido)undecanoylamido)-PEG₁₁-carboxylic Acid (6c)

500 mg of 2-chlorotrityl-(Fmoc-NH-PEG₁₁) resin were reacted with 3.0 mMoles (1.27 g) Fmoc-aminoundecanoic acid in the first synthesis step and 3.0 mMoles (663 mg) of 2,4-dichlorophenoxyacetic acid in the second step according to general description 6. A yellow oily product was isolated. Yield 58%.

¹H-NMR (360 MHz, CDCl₃) δ 1.29 (m, 12H), 1.59 (m, 4H), 2.24 (t, 2H), 2.61 (t, 2H), 3.36 (q, 2H), 3.47 (m, 2H), 3.57 (t, 2H), 3.65 (m, 44H), 3.77 (t, 2H), 4.54 (s, 2H), 6.85 (d, 1H), 7.23 (dd, 1H), 7.42 (d, 1H).

¹³C-NMR (90.6 MHz, CDCl₃) δ 25.4, 26.4, 28.7, 28.8, 28.9, 29.0, 34.5, 35.9, 38.9, 39.2, 66.2, 67.8, 69.3, 69.8, 69.9, 70.0, 70.2, 76.9, 114.4, 123.4, 127.1, 127.7, 129.8, 151.2, 167.2, 173.2, 174.6.

MS-ESI m/z: 1002.487 [M] (calculated for C₄₆Cl₂H₈₀N₂O₁₇: 1002.483).

### Example 11

### Preparation of 6-(2-(2,4-dichlorophenoxy)acetylamido)-PEG₁₁-succinimidylester (7a)

61 μMoles (50 mg) of (6a) were dissolved in a solution of 100 μMoles (11 mg) N-hydroxysuccinimide (NHS) and 100 μMoles (21 mg) N,N′-dicyclohexylcarbodiimide (DCC) in 500 μl anhydrous dioxane. This mixture was stirred over night at RT, 500 μl diethylether were added, the mixture was cooled to 0° C., filtrated and the solvent was removed in vacuo. Yield 82%.

¹H-NMR (360 MHz, CDCl₃) δ 2.83 (s, 4H), 2.90 (t, 2H), 3.57 (t, 2H), 3.64 (m, 46H), 3.85 (t, 2H), 4.53 (s, 2H), 6.85 (d, 1H), 7.22 (dd, 1H), 7.41 (d, 1H).

¹³C-NMR (90.6 MHz, CDCl₃) δ 25.5, 32.1, 38.9, 65.7, 68.3, 69.5, 70.4, 70.5, 70.6, 70.7, 77.2, 114.7, 123.8, 127.3, 128.0, 130.2, 151.7, 166.7, 167.2, 168.9.

MS-ESI m/z: 976.361 [M+AcOH] (calculated for C₃₉Cl₂H₆₂N₂O₁₈: 916.346).

### Preparation of (6-(2-(2,4-dichlorophenoxy)acetylamido)hexylamido)-PEG₁₁-succinimidylester (7b)

53 μMoles (50 mg) of (6b) were dissolved in a solution of 100 μMoles (11 mg) N-hydroxysuccinimide (NHS) and 100 μMoles (21 mg) N,N′-dicyclohexylcarbodiimide (DCC) in 500 μl anhydrous dioxane. This mixture was stirred over night at RT, 500 μl diethylether were added, the mixture was cooled to 0° C., filtrated and the solvent was removed in vacuo. Yield 91%.

¹H-NMR (360 MHz, CDCl₃) δ 1.38 (m, 2H), 1.59 (quin, 2H), 1.69 (quin, 2H), 2.26 (t, 2H), 2.84 (m, 4H), 2.91 (t, 2H), 3.36 (q, 2H), 3.45 (m, 2H), 3.57 (t, 2H), 3.65 (m, 44H), 3.85 (t, 2H), 4.52 (s, 2H), 6.85 (d, 1H), 7.23 (dd, 1H), 7.41 (d, 1H).

¹³C-NMR (90.6 MHz, CDCl₃) δ 25.4, 25.6, 26.2, 29.0, 32.1, 32.4, 38.8, 40.0, 65.7, 68.3, 69.3, 70.1, 70.4, 70.5, 70.6, 70.7, 77.2, 114.8, 123.8, 127.4, 128.0, 130.2, 151.7 166.7, 167.4, 168.9, 171.4.

MS-ESI m/z: 1089.452 [M+AcOH] (calculated for C₄₅Cl₂H₇₃N₃O₁₉: 1029.422).

### Preparation of (6-(2-(2,4-dichlorophenoxy)acetylamido) undecanoylamido)-PEG₁₁-succinimidylester (7c)

50 μMoles (50 mg) of (6c) were dissolved in a solution of 100 μMoles (11 mg) N-hydroxysuccinimide (NHS) and 100 μMoles (21 mg) N,N′-dicyclohexylcarbodiimide (DCC) in 500 μl anhydrous dioxane. This mixture was stirred over night at RT, 500 μl diethylether were added, the mixture was cooled to 0° C., filtrated and the solvent was removed in vacuo. Yield 86%.

¹H-NMR (360 MHz, CDCl₃) δ 1.28 (m, 12H), 1.59 (m, 4H), 2.18 (t, 2H), 2.87 (m, 4H), 2.91 (t, 2H), 3.35 (q, 2H), 3.45 (m, 2H), 3.56 (t, 2H), 3.64 (m, 44H), 3.85 (t, 2H), 4.51 (s, 2H), 6.85 (d, 1H), 7.23 (dd, 1H), 7.42 (d, 1H).

¹³C-NMR (90.6 MHz, CDCl₃) δ 25.4, 25.6, 25.9, 26.8, 29.1, 29.2, 29.3, 29.4, 32.2, 37.5, 39.1, 39.7, 65.7, 68.3, 70.2, 70.5, 70.6, 70.7, 77.2, 114.7, 123.7, 127.4, 128.0, 130.2, 151.6, 166.7, 167.0, 168.9, 169.4.

MS-ESI m/z: 1159.536 [M+AcOH] (calculated for C₅₀Cl₂HB3N₃O₁₉: 1099.500).

### Example 12

### Determination of Hydrolysis Half-Lifes of Active Ester Derivatives

The hydrolysis half-lifes of active ester derivatives in physiological buffer were determined by ¹H-NMR spectroscopy. For this purpose a 50 mM phosphate buffer in deuterium oxide (D₂O, δ9.98%) (deutero, Kastellaun, FRG), pD 7.2, was prepared. The phosphate salts were lyophilized thrice from fresh deuterium oxide. Finally, fresh D₂O was added and the pD value was verified. Stock solutions of active ester derivatives of 2,4-dichlorophenoxyacetic acid (2,4-D) (1b, 3b, or 3d), digoxigenin (Digoxigenin-3-O-methylcarbonyl-ε-aminocaproyl-N-hydroxysuccinimide (DIG-C6-NHS) (Sigma-Aldrich, Taufkirchen, FRG)) or biotin (sulfo-succinimidyl-biotin or sulfo-LC-biotin (KMF Laborchemie, Lohmar, FRG)) (each 15-30 mg/ml, 50 mM) were prepared freshly for each experiment in anhydrous D₆-dimethylsulfoxide (D₆-DMSO, H₂O<0.01%; Euriso-Top, Saarbrucken, FRG). To initiate the hydrolysis reaction 10 μl of these solutions were added to 490 μl of 50 mM deuterated phosphate-buffer, pD 7.2, spiked with 3-(trimethylsilyl)-1-propanesulfonic acid-D₆ sodium salt (TMSPS) (Merck, Darmstadt, FRG) (0.1 mg/ml) as internal reference, resulting in a final concentration of active ester derivatives of 1 mM. The hydrolysis of the active esters was monitored at room temperature by ¹H-NMR spectroscopy with an Avance DRX-600 system (Bruker BioSpin, Rheinstetten, FRG) at 600 MHz. The first ¹H-spectrum was recorded 6-12 min after addition of the phosphate buffer, additional spectra were recorded at about 20, 40, 60, 120, 240, 360 and 1000-1600 min (over night). After the last measurement the pD-value was verified again. The hydrolysis reaction of each active ester derivative was measured in 2-3 independent experiments.

The Bruker software X-WIN-NMR (V 2.5; Bruker BioSpin) was used to acquire and process the data recorded at different time points. For this purpose, signals which were altered in intensity or chemical shift due to deuterolysis and which were not superimposed by signals of other nuclei were integrated and put in relation to the internal standard TMSPS. The concentration of the non-hydrolyzed active ester derivatives was calculated from the change of peak integrals and was plotted versus time. The hydrolysis half-lifes at which 50% of the respective compound was hydrolyzed was determined by nonlinear regression analysis applying the GraphPad Prism Suite (v.4.0.0, GraphPad Software, San Diego, Calif., USA) (Table 1).

TABLE 1
Hydrolysis half-lifes of active ester derivatives of 2,4-dichlorophenoxyacetic
acid (2,4-D), digoxigenin (DIG) or biotin.

| | Hydrolysis | 95% Confi- | Goodness |
|---|---|---|---|
| | half-life | dence interval | of Fit |
| Active ester derivative | [min] | [min] | [R²-value] |
| 2,4-D-NHS^{(a)} (1b) | 25 | 23-28 | 0.99 |
| 2,4-D-C6^{(b)}-sulfo-NHS (3b) | 231⁽*⁾ | 216-248 | 0.99 |
| 2,4-D-C11^{(c)}-sulfo-NHS (3d) | n.a. | n.a. | n.a. |
| DIG-C6^{(b)}-NHS | 248⁽*⁾ | 234-265 | 0.99 |
| Sulfo-NHS-biotin | 62 | 53-74 | 0.95 |
| Sulfo-NHS-LC^{(b)}-biotin | 101 | 85-122 | 0.93 |

^{(a)}N-hydroxysuccinimidylester;
^{(b)}aminohexanoyl-(C6)-spacer;
^{(c)}aminoundecanoyl-(C11)-spacer.
Hydrolysis half-lifes as well as the 95% confidence intervals were determined by nonlinear regression analysis;
⁽*⁾hydrolysis half-lifes of (3b) and DIG-C6-NHS are significantly above hydrolysis half-lifes of all other active esters (one-way ANOVA, Bonferroni post hoc test, P < 0.001), but do not differ from each other (one-way ANOVA, Bonferroni post hoc test, P > 0.05).
n.a.: not applicable as (3d) aggregated under the conditions used in the experiment.

### Example 13

### Labelling of a Proteinaceous Substrate (>20000 Da) with 2,4-dichlorophenoxyacetic Acid Active Ester Derivatives, Active Esters of Other Labels and Detection Thereof

Stock solutions of 65-100 mg/ml (120-330 mM) active ester of the labelling compounds (Table 2) were prepared freshly in anhydrous dimethylsulfoxide (DMSO) for each labelling experiment. A stock solution of 135 mg/ml (3 mM) ovalbumin (chicken egg, MW 45000; Merck Biosciences, Bad Soden, FRG) in 100 mM sodium tetraborate, pH 8.2, was prepared, snap-frozen in liquid nitrogen and stored at −80° C. For labelling 100 nMoles ovalbumin (representing 2 μMoles amino functions) were reacted with 1.5 μMoles of active ester in a final volume of 300 μl 100 mM sodium tetraborate, pH 8.2, containing no more than 5% (v/v) DMSO in any experiment. The solutions were mixed on an end-to-end mixer for 180 min at RT and the labelling reactions were terminated by addition of 1.5 mmoles glycine in sodium tetraborate buffer. Mixing was continued overnight at 4° C. before the solutions were dialyzed against sodium tetraborate buffer. The concentrations of the labelled protein were determined by standard assays and serial dilutions of the stock solutions were prepared. Equal amounts of the diluted solutions were applied (either 16,000-31.2 pg conjugate/dot for biotin-SLC ovalbumin conjugates or 1,600-3.12 μg conjugate/dot for all other conjugates) onto nitrocellulose membranes (4.8×3 cm, 0.2 μm pore size, Whatman, Schleicher & Schuell, Dassel, FRG) and the membranes were allowed to air-dry before they were stored at −20° C. Immediately before continuing the experiment the membranes were thawed and washed 3× with Dulbecco's phospate-buffered saline, pH 7.4 (D-PBS: 2.7 mM KCl, 1.5 mM KH₂PO₄, 136 mM NaCl, 8.1 mM Na₂HPO₄). All subsequent treatments of the membranes were carried out in tight fitting trays. The membranes were blocked with 1% (w/v) casein (Hammarsten grade, BDH, Poole, UK) in D-PBS (Casein-PBS) for 30 min at RT. Blocked membranes were incubated for 90 min at RT with 4 ml (0.28 ml/cm² membrane) diluted monoclonal antibodies against 2,4-dichlorophenoxyacetic acid or monoclonal antibodies against DIG or with AlexaFluor680-labelled streptavidin (for working concentrations see Table 3) in Casein-PBS and washed 3× with D-PBS. The membranes treated with anti-2,4-dichlorophenoxyacetic acid or anti-DIG antibodies were further incubated for 90 min at RT with 3 ml (0.21 ml/cm² membrane)₂ μg/ml secondary antibody (goat anti-mouse AlexaFluor680, Invitrogen, Carlsbad, Calif., USA) in Casein-PBS while the membranes treated with streptavidin AlexaFluor680 were kept in Casein-PBS for that time period. After intense washing of the membranes with D-PBS fluorescence was read out on a fluorescence imager (Odyssey Infrared Imager, LI-COR Biosciences, Lincoln, Nebr., USA) and quantitated with an appropriate image analysis software (Odyssey Software v.1.2). Statistical analysis of the data was carried out with the GraphPad Prism Suite (v.4.0.0, GraphPad Software, San Diego, Calif., USA). The lower detection limits for each conjugate are summarized in Table 5.

TABLE 2
Active esters of different labels used
in polypeptide labelling experiments

| Active | | |
|---|---|---|
| ester of | | Vendor (if ap- |
| label | Chemical name | plicable) |
| Biotin | Sulfo-Succinimidyl-(+)-biotin | Molecular Bio- |
| | | sciences |
| LC-Biotin | Sulfo-LC-(+)-Biotin | Molecular Bio- |
| | | sciences |
| SLC- | SLC-NHS-(+)-Biotin | Molecular Bio- |
| Biotin* | | sciences |
| LC-DIG* | Digoxigenin-3-O-methylcarbonyl-ε- | Roche |
| | aminocaproyl-N-hydroxysuccinimide | Diagnostics |
| 2,4-D* | 2-(2,4-dichlorophenoxy)acetic acid-N- | — |
| | hydroxysuccinimidyl-ester | |
| 2,4-D-Ahx | 6-(2-(2,4-dichlorophenoxy)acetylamido)- | — |
| | hexanoic acid-N-hydroxysulfosuccinimidyl- | |
| | ester-sodium salt | |
| 2,4-D-Aun | 11-(2-(2,4-dichlorophenoxy)acetylamido)- | — |
| | undecanoic acid-N-hydroxysulfo- | |
| | succinimidyl-ester-sodium salt | |

*= these derivatives do not carry a sulfo-group.

TABLE 3
Primary detection reagents for the different labels

| | | | Working con- | |
|---|---|---|---|---|
| Label | Primary detection reagent | Clone | centration | |
| 2,4-D | Mouse anti-2,4-D (M. Franek, | E2/G2 | 1 | μg/ml |
| | Brno, CZ) | E4/C2 | 1 | μg/ml |
| | | F6/C10 | 1 | μg/ml |
| | | B7 | 1 | μg/ml |
| DIG | Mouse anti-Digoxigenin (Roche | 1.71.256 | 1 | μg/ml |
| | Diagnostics) | | | |
| Biotin | Streptavidin AlexaFluor680 | | 438 | ng/ml |
| | (Invitrogen) | | | |

### Example 14

### Labelling of a Glycoprotein with 2,4-dichlorophenoxyacetic acid-hydrazide Derivatives, Hydrazide Derivatives of Other Labels and Detection Thereof

A stock solution of 10 μg/ml of a glycoprotein (porcine mucin, Sigma-Aldrich, Taufkirchen, FRG) was prepared in Dulbecco's phosphate buffered saline, pH 7.4 (D-PBS: 2.7 mM KCl, 1.5 mM KH₂PO₄, 136 mM NaCl, 8.1 mM Na₂HPO₄), snap-frozen in liquid nitrogen and stored at −80° C. For labelling the glycoprotein carbohydrates the thawed stock solution was serially diluted, equal amounts of the diluted solutions were applied (2000-1 pg glycoprotein/dot) onto nitrocellulose membranes (4.8×3 cm, 0.2 μm pore size, Whatman, Schleicher & Schuell, Dassel, FRG), allowed to air-dry and the membranes were stored at −20° C. Immediately before continuing the experiment the membranes were thawed and were washed 3× with Dulbecco's phospate-buffered saline, pH 7.4 (D-PBS: 2.7 mM KCl, 1.5 mM KH₂PO₄, 136 mM NaCl, 8.1 mM Na₂HPO₄). All subsequent treatments of the membranes were carried out in tight fitting trays. For oxidation or the carbohydrate moieties of the glycoprotein the membranes were incubated for 20 min at RT in 3 ml (0.21 ml/cm² membrane) 10 mM sodium m-periodate (Sigma-Aldrich, Taufkirchen, FRG) in 100 mM sodium acetate buffer (pH 5). They were washed another 3× in D-PBS, incubated each for 60 min at RT with 3 ml (0.21 ml/cm² membrane) of 2.5 μM labelling-hydrazide (Table 4) in 100 mM sodium acetate buffer (pH 5), washed again 3× in D-PBS and blocked with 1% (w/v) Casein (Hammarsten grade, BDH, Poole, UK) in D-PBS (Casein-PBS) for 30 min at RT. Blocked membranes were incubated for 90 min at RT with 4 ml (0.28 ml/cm² membrane) diluted monoclonal antibodies against 2,4-dichlorophenoxyacetic acid or monoclonal antibodies against DIG or with AlexaFluor680-labelled streptavidin (for working concentrations see Table 3) in Casein-PBS and washed 3× with D-PBS. The membranes treated with anti-2,4-dichlorophenoxyacetic acid or anti-DIG antibodies were further incubated for 90 min at RT with 3 ml (0.21 ml/cm² membrane) 2 μg/ml secondary antibody (goat anti-mouse AlexaFluor680, Invitrogen, Carlsbad, Calif., USA) in Casein-PBS while the membranes treated with streptavidin AlexaFluor680 were kept in Casein-PBS for that time period. After intense washing with D-PBS fluorescence was read out on a fluorescence imager (Odyssey Infrared Imager, LI-COR Biosciences, Lincoln, Nebr., USA) and quantitated with an appropriate image analysis software (Odyssey Software v.1.2). Statistical analysis of the data was carried out with the GraphPad Prism Suite (v.4.0.0, GraphPad Software, San Diego, Calif., USA). The lower detection limits for each conjugate are summarized in Table 5.

TABLE 4
Hydrazides of different labels used in
glycoprotein labelling experiments

| Hydrazides | | |
|---|---|---|
| of different | | Vendor (if |
| labels | Chemical name | applicable) |
| Biotin | (+)-Biotinoylhydrazide | Molecular |
| | | Biosciences |
| LC-Biotin | LC-(+)-Biotinoylhydrazide | Molecular |
| | | Biosciences |
| SLC-Biotin | SLC-(+)-Biotinoylhydrazide | Molecular |
| | | Biosciences |
| LC-DIG | Digoxigenin-3-O-succinyl-aminohexanoyl- | Roche Diag- |
| | hydrazide | nostics |
| 2,4-D | 2-(2,4-dichlorophenoxy)acetic acid- | — |
| | hydrazide | |
| 2,4-D-Ahx | 6-(2-(2,4-dichlorophenoxy)acetylamido)- | — |
| | hexanoic acid-hydrazide | |
| 2,4-D-Aun | 11-(2-(2,4-dichlorophenoxy)acetylamido)- | — |
| | undecanoic acid-hydrazide | |

TABLE 5
Detection limits of 2,4-dichlorophenoxyacetic acid
derivative-labelled proteins in comparison to analogous
digoxigenin- or biotin-labelled conjugates

| | | | Detection limit [pg] of proteins after | |
|---|---|---|---|---|
| | Primary | | labelling with | |
| | antibody | | Active ester | Hydrazide |
| Label | (clone) | Spacer | derivatives^{(a)} | derivatives^{(b)} |
| 2,4-D^{(c)} | E2/G2 | — | 161.5 ± 41.3 | 114.6 ± 10.4 |
| | | C6^{(d)} | 388.4 ± 72.1 | 24.8 ± 4.4^{(e)} |
| | | C11^{(f)} | 625.0 ± 125.0^{(g)} | 51.9 ± 13.8^{(g)} |
| | E4/C2 | — | 261.2 ± 76.3 | 160.7 ± 23.1 |
| | | C6^{(d)} | 70.3 ± 17.9 | 25.1 ± 8.0^{(e)} |
| | | C11^{(f)} | 415.2 ± 94.2^{(g)} | 28.7 ± 9.4^{(g)} |
| | F6/C10 | — | 236.3 ± 62.6 | 114.6 ± 10.4 |
| | | C6^{(d)} | 253.9 ± 60.4 | 2.7 ± 0.4^{(h)} |
| | | C11^{(f)} | 386.7 ± 110.5^{(g)} | 13.7 ± 1.3^{(g)} |
| | B7 | — | 323.7 ± 65.7 | 100.4 ± 28.5⁽ⁱ⁾ |
| | | C6^{(d)} | 352.7 ± 71.8 | 10.9 ± 1.8^{(h)} |
| | | C11^{(f)} | 625.0 ± 178.9^{(g)} | 15.6 ± 0.0^{(g)} |
| DIG^{(j)} | 1.17.256 | — | n.a. | n.a. |
| | | C6^{(d)} | 171.9 ± 55.4 | 80.4 ± 11.5 |
| | | C11^{(f)} | n.a. | n.a. |
| Biotin | —^{(k)} | — | 246.5 ± 67.2 | 270.1 ± 64.2 |
| | | C6^{(d)} | 248.0 ± 66.6 | 49.1 ± 6.3 |
| | | C11^{(f)} | 39063.0 ± 5508.0 | 114.6 ± 10.4 |

^{(a)}Ovalbumin was labelled with different active esters. Conjugates were serially diluted and immobilized on nitrocellulose membrane as described.
^{(b)}The glycosylated protein mucin was serially diluted, immobilized on nitrocellulose, oxidized with periodate and labelled with different hydrazides as described. The used reagents either contained
^{(c)}2,4-dichlorophenoxyacetic acid (2,4-D),
^{(j)}digoxigenin (DIG) or biotin as label and either no (—) aliphatic spacer or
^{(d)}aminohexanoic acid (C6) or
^{(f)}aminoundecanoic acid (C11) as spacer. 2,4-dichlorophenoxyacetic acid- and DIG-labels were detected by specific primary antibodies and AlexaFluor680 labelled secondary antibody whereas
^{(k)}biotin-labels were visualized with streptavidin-AlexaFluor680. The detection limits (arithmetic means ± SEM) above cut-off are given in pg. The cut-off was calculated according to the procedure described by Frey et al. (Frey A, Di Canzio J, Zurakowski D (1998). A statistically defined endpoint titer determination method for immunoassays. J Immunol Methods. 221: 35-41.).
⁽ⁱ⁾Detection limits for 2,4-dichlorophenoxyacetic acid labels that were significantly lower than those after labelling with biotin,
^{(e)}after labelling with C6-spacered biotin, or
^{(h)}after labelling with C6-spacered biotin or DIG, or
^{(g)}after labelling with C11-spacered biotin (P < 0.05, One-way ANOVA, Bonferroni Post hoc test).
n.a.: not applicable, as the labelling reagent is not commercially available.

### Example 15

### Labelling of a Polypeptidic Substrate (<20000 Da) with 2,4-dichlorophenoxyacetic Acid Active Ester Derivatives, Active Esters of Other Labels and Detection Thereof

Stock solutions of active esters of the labelling compounds summarized in Table 2 (4.2-9.2 mg/ml, 14 mM) as well as labelling compounds (7a), (7b) and (7c) (approximately 15 mM) were prepared freshly in anhydrous dimethylsulfoxide (DMSO) for each labelling experiment. Likewise, a stock solution of insulin (200 μg/ml, 35 μM; bovine insulin, MW 5733,49; Sigma-Aldrich, Taufkirchen, FRG) in 50 mM phosphate buffer, pH 7.2, 0.02 mM ethylenediaminetetraacetic acid (EDTA) (PBS/EDTA) was prepared. For labelling, insulin (35 nMoles, representing 105 nMoles amino functions (2 NH₂-termini (chain A and B), 1 lysine side chain)) was reacted with active esters (350 nMoles) in a final volume of 1.25 ml PBS/EDTA, containing no more than 2% (v/v) DMSO in any experiment. The solutions were mixed on an end-to-end mixer for 90 min at RT and the labelling reactions were terminated by addition of glycine (1 mMole) in PBS/EDTA. Mixing was continued for 60 min at RT before the solution was dialyzed against PBS/EDTA (MWCO 2,000). After another dialysis against water, the heterogeneity of the samples and the molecular masses of the products were determined in MALDI-TOF-MS analyses (Bruker-Reflex II instrument; Bruker-Daltonik, Bremen, FRG). The mean labelling degree was calculated as described elsewhere (Olivier V, Meisen I, Meckelein B, Hirst T R, Peter-Katalinic J, Schmidt M A, Frey A (2003). Influence of targeting ligand flexibility on receptor binding of particulate drug delivery systems. Bioconjug Chem. 14:1203-8.) (Table 6).

The concentrations of the labelled polypeptides were determined by standard assays and serial dilutions of the solutions were prepared in PBS/EDTA. Equal amounts of the diluted solutions (1,000-0.12 ng conjugate/well, each in 50 μl PBS/EDTA) were applied onto 96-well polystyrene filterplates equipped with polyvinylidene fluoride (PVDF) membrane bottoms (Corning, Corning, N.Y., USA), which had been pre-activated with 70% (v/v) ethanol and washed with phosphate buffer. The plates were incubated for 30 min at room temperature before the solutions were sucked through the membranes with a MultiScreen vacuum manifold (Millipore, Schwalbach, FRG). Plates were washed 3× with 200 μl Dulbecco's phosphate-buffered saline, pH 7.4 (D-PBS: 2.7 mM KCl, 1.5 mM KH₂PO₄, 136 mM NaCl, 8.1 mM Na₂HPO₄), containing 0.1% (v/v) Tween20, by using the vacuum manifold and three times with 300 μl/well D-PBS with an automated plate washer. Nonspecific binding sites were blocked with 200 μl/well 2% (w/v) caseinate in D-PBS (Caseinate-PBS) for 30 min at RT and the solutions were removed. Blocked plates were again washed as described above before they were incubated for 30 min at RT with 50 μl/well of diluted monoclonal antibodies against 2,4-dichlorophenoxyacetic acid or monoclonal antibodies against DIG (each 1 μg/ml) or of horseradish peroxidase (HRP)-labelled streptavidin (0.44 μg/ml; Vector Labs, Burlingame, Calif., USA) in Caseinate-PBS. After another washing cycle (4× each) the plates treated with anti-2,4-dichlorophenoxyacetic acid or anti-DIG antibodies were further incubated for 30 min at RT with 50 μl/well of 2 μg/ml HRP-labelled goat anti-mouse IgG antibody (Southern Biotechnology, Birmingham, Ala., USA) in Caseinate-PBS while the membranes treated with streptavidin-HRP were kept in Caseinate-PBS for that time period. After the plates were washed another 6× with PBST with the vacuum manifold and 6× with D-PBS with the plate washer, color was developed at room temperature in the dark with 75 μl/well of a highly sensitive tetramethylbenzidine-based substrate reagent (1 mM 3,3′,5,5′-tetramethylbenzidine, 3 mM H₂O₂ in 200 mM potassium citrate, pH 4.0; (Frey A, Meckelein B, Externest D, Schmidt M A (2000). A stable and highly sensitive 3,3′,5,5′-tetramethylbenzidine-based substrate reagent for enzyme-linked immunosorbent assays. J Immunol Methods. 233:47-56.)). The reaction was terminated after 30 min with 125 μl/well 1 M sulfuric acid. The supernatants (175 μl/well) were transferred to a non-binding 96-well polystyrene plate (Corning) and the absorbances at 450 and 405 nm were determined with a Versamax microtiter plate reader (Molecular Devices, Sunnyvale, Calif., USA). Statistical analysis of the data was carried out with the GraphPad Prism Suite (v.4.0.0, GraphPad Software, San Diego, Calif., USA). The lower detection limits for each conjugate are summarized in Table 7 and Table 8.

TABLE 6
Mean degree of derivatization of insulin labelled with
active ester derivatives of 2,4-dichlorophenoxyacetic
acid (2,4-D), digoxigenin (DIG) or biotin.

| | Mean labelling de- | Coefficient |
|---|---|---|
| | gree [Mole label: | of variation |
| Active ester derivative | Mole insulin] | [%] |
| 2,4-D-NHS^{(a)} (1b) | 0.52 | 38.40 |
| 2,4-D-C6^{(b)}-sulfo-NHS (3b) | 1.61 | 0.60 |
| 2,4-D-C11^{(c)}-sulfo-NHS (3d) | 0.38 | 4.00 |
| DIG-C6^{(b)}-NHS | 1.50 | 6.70 |
| Sulfo-NHS-biotin | 1.79 | 0.60 |
| Sulfo-NHS-LC^{(b)}-biotin | 1.72 | 5.2 |
| NHS-SLC^{(c)}-biotin | 0.08 | 12.5 |

^{(a)}N-hydroxysuccinimidylester;
^{(b)}aminohexanoyl-(C6)-spacer;
^{(c)}aminoundecanoyl-(C11)-spacer.
The mean labelling degree was determined in three independent labelling experiments according to Olivier et al. (Olivier V, Meisen I, Meckelein B, Hirst T R, Peter-Katalinic J, Schmidt M A, Frey A (2003). Influence of targeting ligand flexibility on receptor binding of particulate drug delivery systems. Bioconjug Chem. 14: 1203-8.).

TABLE 7
Detection limits of 2,4-dichlorophenoxyacetic acid derivative-labelled proteins
in comparison to digoxigenin or biotin derivative-labelled conjugates

| | Primary | | | |
|---|---|---|---|---|
| | antibody | | Detection limit [pg] of | Detection limit [fMole] |
| Label | (clone) | Spacer | labelled protein^{(a)} | of label |
| 2,4-D^{(b)} | E2/G2 | — | 197,916.7 ± 42,020.3 | 17,950.1 ± 3,811.0 |
| | | C6^{(c)} | 915.6 ± 61.0^{(d)} | 257.1 ± 17.1^{(d)} |
| | | C11^{(e)} | 160.2 ± 25.6^{(f)} | 10.6 ± 1.7^{(f)} |
| | E4/C2 | — | 9,114.6 ± 1,210.1 | 826.7 ± 109.8^{(g)} |
| | | C6^{(c)} | 42.0 ± 7.5^{(d)} | 11.8 ± 2.1^{(d)} |
| | | C11^{(e)} | 10.9 ± 1.6^{(f)} | 0.7 ± 0.1^{(f)} |
| | F6/C10 | — | 180,555.6 ± 21,960.3 | 16,375.5 ± 1,991.7 |
| | | C6^{(c)} | 1,519.1 ± 456.3^{(d)} | 426.6 ± 128.1^{(d)} |
| | | C11^{(e)} | 133.5 ± 27.0^{(f)} | 8.9 ± 1.8^{(f)} |
| | B7 | — | 173,611.1 ± 25,038.6 | 15,745.7 ± 2,270.8 |
| | | C6^{(c)} | 1,464.9 ± 369.1^{(d)} | 411.4 ± 103.7^{(d)} |
| | | C11^{(e)} | 113.4 ± 8.7^{(f)} | 7.5 ± 0.6^{(f)} |
| DIG^{(h)} | 1.17.256 | — | n.a. | n.a. |
| | | C6^{(c)} | 21,701.4 ± 3,129.8 | 5,677.5 ± 818.8 |
| | | C11^{(e)} | n.a. | n.a. |
| Biotin | —⁽ⁱ⁾ | — | 62,500.0 ± 15,343.4 | 19,512.6 ± 4,790.2 |
| | | C6^{(c)} | 33,854.2 ± 6,107.3 | 10,156.0 ± 1,832.1 |
| | | C11^{(e)} | 1,277,778.0 ± 237,333.4 | 17,829.0 ± 3,311.5 |

^{(a)}Insulin was labelled with different active esters. Conjugates were serially diluted and immobilized on PVDF membrane as described. The used reagents either contained
^{(b)}2,4-dichlorophenoxyacetic acid (2,4-D),
^{(h)}digoxigenin (DIG) or biotin as label and either no (—) aliphatic spacer or
^{(c)}aminohexanoic acid (C6) or
^{(e)}aminoundecanoic acid (C11) as spacer. 2,4-Dichlorophenoxyacetic acid- and DIG-labels were detected by specific primary antibodies and HRP-labelled secondary antibody whereas
⁽ⁱ⁾biotin-labels were visualized with streptavidin-HRP. The lower detection limits (LDL) (arithmetic means ± SEM) above cut-off are given in pg labelled protein and converted to femtomole label: LDL (fMole label) = [LDL(pg protein)/MWᵢₙₛᵤₗᵢₙ(5.7335 pg/fMole)] * labelling degree (Table 6). The cut-off was calculated according to the procedure described by Frey et al. (Frey A, Di Canzio J, Zurakowski D (1998). A statistically defined endpoint titer determination method for immunoassays. J Immunol Methods. 221: 35-41.).
^{(g)}Detection limits for 2,4-dichlorophenoxyacetic acid labels that are significantly lower than those after labelling with biotin (ANOVA, Bonferroni Post hoc test, P < 0.001) or
^{(d)}after labelling with C6-spacered biotin (P < 0.001) and DIG (P < 0.01), or
^{(f)}after labelling with C11-spacered biotin (P < 0.001).
n.a.: not applicable, as the labelling reagent is not commercially available.

TABLE 8
Detection limits of proteins labelled with 2,4-dichlorophenoxyacetic
acid derivatives containing oligoethyleneglycol moieties

| Primary | | |
|---|---|---|
| antibody | Labelling | Detection limit [pg] of |
| (clone) | derivative | labelled protein^{(a)} |
| E2/G2 | (7a) | 3,906.3 ± 0.3 |
| | (7b) | 1,099.7 ± 307.5^{(b)} |
| | (7c) | 68.8 ± 53.5^{(b)} |
| E4/C2 | (7a) | 732.9 ± 109.1 |
| | (7b) | 312.3 ± 58.0^{(b)} |
| | (7c) | 45.8 ± 15.3^{(b)} |
| F6/C10 | (7a) | 6,341.4 ± 3,120.3 |
| | (7b) | 3,905.0 ± 1,381.2 |
| | (7c) | 366.1 ± 122.0 |
| B7 | (7a) | 6,508.8 ± 1,301.2 |
| | (7b) | 2,541.5 ± 586.5^{(b)} |
| | (7c) | 733.2 ± 244.4^{(b)} |

^{(a)}Insulin was labelled with different 2,4-dichlorophenoxyacetic acid (2,4-D)-active ester derivatives which contained oligoethyleneglycol moieties in the spacer (7a, 7b or 7c). Conjugates were serially diluted and immobilized on PVDF membrane as described. 2,4-Dichlorophenoxyacetic acid-labels were detected by specific primary antibodies and HRP-labelled secondary antibody. The lower detection limits (LDL) (arithmetic means ± SEM) above cut-off are given in pg labelled protein. The cut-off was calculated according to the procedure described by Frey et al. (Frey A, Di Canzio J, Zurakowski D (1998). A statistically defined endpoint titer determination method for immunoassays. J Immunol Methods. 221: 35-41.).
^{(b)}Detection limits for 2,4-dichlorophenoxyacetic acid labels that are significantly lower than those after labelling with (7a) (ANOVA, Bonferroni Post hoc test P < 0.05).

### Example 16

### In-Sequence-Labelling of a Synthetic Peptide with 2,4-dichlorophenoxyacetic Acid Derivatized N-alpha-(9-fluorenylmethyloxycarbonyl)-L-lysines and Detection Thereof

18mer oligopeptides, containing the 16mer aminoterminal fragment of ovalbumin, were SPOT-synthesized on cellulose membranes according to Frank (Frank R (1992). Spot synthesis: an easy technique for the positionally addressable, parallel chemical synthesis on a membrane support. Tetrahedron. 48:9217-9232). Briefly, cellulose membranes were derivatized with 0.02 ml/cm² fluorenylmethoxycarbonyl-(Fmoc)-protected proline (0.2 M Fmoc-proline, 0.46 M methylimidazol and 0.26 M diisopropylcarbodiimide (DICD) in N,N-dimethylformamide (DMF)). Excess hydroxyl groups were blocked for 24 h with 15 ml (0.13 ml/cm² membrane) 2% (v/v) acetic acid anhydride in DMF and the membranes were washed 3× with DMF. Fmoc cleavage was done twice by 5 min incubation with 10 ml (0.09 ml/cm² membrane) 20% (v/v) piperidine in DMF, membranes were washed again 5× with DMF and the presence of primary amino groups on the membrane was verified by staining for 10 min at RT with 15 ml (0.13 ml/cm² membrane) 0.01% (w/v) bromophenolblue in DMF. Subsequently membranes were washed 3× with 100% ethanol and air dried. These washing steps and the staining were repeated between all synthesis cycles. For capping of unreacted amino functions incubation with acetic acid anhydride was shortened to 20 min after the third synthesis cycle. Synthesis cycles were carried out with a pipetting robot (ASP 222, Intavis Bioanalytical Instruments AG, Koln, FRG). The robot applied 0.1 μl of a 0.2 M solution of Boc-(tert-butoxycarbonyl)-lysine-(Fmoc)-OH, containing 0.35 M hydroxybenzotriazole (HOBt) and 0.25 M DICD in N-methylpyrrolidone, on defined areas, so-called SPOTs. In subsequent synthesis cycles 0.2 μl of 0.2 M solutions of N-alpha-Fmoc-protected amino acids with protected side chains if applicable (tert.-butyl (tBu) for serine, threonine, tyrosine, glutamic acid, aspartic acid; trityl for asparagine, glutamine, histidine; t-butyloxycarbonyl (Boc) for lysine, tryptophane; 2,2,4,6,7-pentamethyldihydrobenzofurane-5-sulfonyl (Pbf) for arginine; acetamido methyl (Acm) for cysteine)), containing 0.35 M HOBt in N-methylpyrrolidone and 0.25 M DICD, were applied on these SPOTs. 30 min before the respective coupling solutions were used, the amino acids therein were converted into the respective active esters by addition of 1.25 Mole DICD per Mole amino acid. The mixture was reacted for 30 min at RT and centrifuged to remove precipitates. Coupling of each amino acid was repeated 3 times and a minimum of 40 min reaction time was allowed in each round. 18mer peptides labelled with 2,4-dichlorophenoxyacetic acid derivatized N-alpha-(9-fluorenylmethyloxycarbonyl)-L-lysines (2c), (4b) or (4d) were synthesized this way.

Protection groups of the side chains (except Acm) were removed by two incubations of the membranes for 1 h each with 10 ml (0.09 ml/cm² membrane) 50% (v/v) trifluoroacetic acid, 2% (v/v) water and 3% (v/v) triisobutylsilane in dichloromethane (DCM). Subsequently, membranes were washed 4× with DCM, 4× with 0.1% (v/v) HCl, 50% (v/v) methanol in water and finally 4× with 1 M acetic acid (pH 1.9). Membranes were desiccated over night. The peptide SPOTs were punched out and transferred to 2 ml polypropylene tubes. To cleave the peptides from the membrane 500 μl of 0.1 M triethylammoniumacetate (TEAA), 20% (v/v) ethanol, pH 7.5, in water were added to each membrane piece. The pieces were incubated over night, the supernatant was transferred to a fresh 2 ml tube and the cleavage reaction was repeated for another 2 h. Both peptide solutions were pooled and the solvent was removed in vacuo. The dried peptides were dissolved in 1.5 ml 10 mM sodium phosphate buffer, pH 7.0, 10 mM NaCl (L-PBS)×0.005% (w/v) Tween 20, snap-frozen in liquid N₂ and stored at −80° C. Scheme a-c gives an overview of the synthesized peptides:

### a) Amino-terminus: Ac-x-GSIGAASMEFCFDVFK-y-z: Carboxy-terminus

### b) Amino-terminus: Ac-y-GSIGAASMEFCFDVFK-x-z: Carboxy-terminus

### c) Amino-terminus: Ac-GSIGAAS-y-MEFCFDVFK-x-z: Carboxy-terminus

(Ac=acetyl, x=lysine(biotin), y=derivatives 2c, 4b or 4d, z=diketopiperazine moiety, single letter code for amino acids)

For the detection of the labelled peptides 96-well high-bind microplates (Corning, Corning, N.Y., USA) were coated overnight at 4° C. with 75 μl/well of 50 ng/ml monoclonal anti-2,4-dichlorophenoxyacetic acid antibodies (clones E2/G2, E4/C2, F6/C10 or B7) in L-PBS. Plates were washed 3× with Dulbecco's phosphate buffered saline, pH 7.4 (D-PBS: 2.7 mM KCl, 1.5 mM KH₂PO₄, 136 mM NaCl, 8.1 mM Na₂HPO₄) and blocked for 3-4 h at RT with 1% (w/v) casein (Hammarsten grade, BDH, Poole, UK) in D-PBS (Casein-PBS). Plates were washed 4× with D-PBS and 75 μl of solutions of serially diluted labelled peptides were added (1:750-1:1,536,000 in D-PBS). The peptides were allowed to bind for 2.5 h at RT before the plates were washed 4× with D-PBS. For detection of the bound peptides plates were incubated with 75 μl/well 1 μg/ml horseradish-labelled streptavidin in Casein-PBS for 60 min at RT (Vector Laboratories, Burlingame, Calif., USA). The plates were washed 6× with D-PBS and color was allowed to develop for 30 min after addition of 75 μl/well of 3,3′,5,5′-tetramethylbenzidine-hydrogen peroxide-substrate (Frey A, Meckelein B, Externest D, Schmidt M A (2000). A stable and highly sensitive 3,3′,5,5′-tetramethylbenzidine-based substrate reagent for enzyme-linked immunosorbent assays. J Immunol Methods. 233:47-56). Color development was terminated by addition of 125 μl/well 1 M sulfuric acid and absorption was determined with a microplate reader (Versamax, Molecular Devices, Sunnyvale, Calif., USA) at 450 nm. The binding performance of peptides labelled with different 2,4-dichlorophenoxyacetic acid derivatized N-alpha-(9-fluorenylmethyloxycarbonyl)-L-lysines is depicted in FIG. 3.

### Example 17

Reversible labelling of a peptide with 2-(2-(2,4-dichlorophenoxy)-1-hydroxyethylidene)-5,5-dimethylcyclohexane-1,3-dione (2,4-D-dimedone) and detection thereof

A 15mer fragment of ovalbumin (NH₂-GSIGAASMEFCFDCF-COOH), was SPOT-synthesized according to example 10. Unlike example 10 the peptide was immobilized to the membrane by the non-cleavable linker, beta-alanylalanine (beta-A) (NH₂-peptide-COOH-beta-A-membrane). Subsequent to removal of the N-terminal Fmoc-protection group the peptide was derivatized with 0.2 μl of 0.2 M 2,4-D-dimedone (1c) in N-methylpyrrolidone. Side chain protection groups were removed as described, the membrane was washed 3× with Dulbecco's phosphate buffered saline, pH 7.4 (D-PBS: 2.7 mM KCl, 1.5 mM KH₂PO₄, 136 mM NaCl, 8.1 mM Na₂HPO₄) and blocked for 5 h at RT with 1% (w/v) casein (Hammarsten grade, BDH, Poole, UK) in D-PBS (Casein-PBS). The 2,4-dichlorophenoxyacetic acid label was detected with 4 ml (2 ml/cm² membrane) 1 μg/ml biotinylated anti-2,4-dichlorophenoxyacetic acid antibody (clone E4/C2; biotinylated with 15-([biotinoyl]amino)-4,7,10,13-tetraoxapentadecanoic acid-N-hydroxysuccinimidyl ester (NHS-PEO4-Biotin) (Uptima via KMF Laborchemie, Lohmar, FRG) according to the manufacturer's instructions) in Casein-PBS overnight at 4° C. followed by 3 ml (1.5 ml/cm² membrane) 250 ng/ml AlexaFluor680 labelled streptavidin (Invitrogen, Carlsbad, Calif., USA) in Casein-PBS for 90 min at RT. After each incubation with antibodies or streptavidin the membrane was washed 6× for 10 min at RT with D-PBS. Fluorescence was quantitated on a fluorescence imager with appropriate software (Odyssey Infrared Imager & Odyssey software V1.2, LI-COR Biosciences, Lincoln, Nebr., USA) and the 2,4-dichlorophenoxyacetic acid label was removed by treating the membrane with 4 ml (2 ml/cm² membrane) 5% (v/v) hydrazine monohydrate in D-PBS for 5, 20 or 60 min at RT. After each incubation the membrane was washed 3× with D-PBS and residual fluorescence was quantitated again. After complete cleavage of the 2,4-dichlorophenoxyacetic acid label the membrane was washed with DMF and the peptide was aminoterminally acetylated with 4 ml (2 ml/cm² membrane) 2% (v/v) acetanhydride in DMF. The membrane was washed intensively with DMF, followed by ethanol and subsequently air-dried. After blocking for 5 h at RT with Casein-PBS, the membrane was incubated with 4 ml (2 ml/cm² membrane) of 1 μg/ml monoclonal mouse anti-ovalbumin antibody (reactive with the 15mer fragment of ovalbumin synthesized herein; Sigma-Aldrich, Taufkirchen, FRG) in Casein-PBS over night at 4° C. followed by 4 ml (2 ml/cm² membrane) of 2 μg/ml goat anti-mouse IgG AlexaFluor680 labelled secondary antibody (Invitrogen, Carlsbad, Calif., USA) in Casein-PBS for 90 min at RT. After each incubation with antibodies the membrane was washed 6× for 10 min at RT with D-PBS. Fluorescence was quantitated as described above. The results are summarized in FIG. 4.

### Example 18

### Immunohistochemical Detection of a 2,4-dichlorophenoxyacetic Acid (2,4-D) Labelled Substrate Molecule

Wheat germ agglutinin (WGA), a lectin recognizing N-acetylglucosamine as receptor sugar, was labelled with active ester derivative (3b). A stock solution of WGA (10 mg/ml, 0.3 mM) in 100 mM sodium tetraborate, pH 8.2, was prepared, snap-frozen in liquid nitrogen and stored at −80° C. A stock solution of (3b) in anhydrous dimethylsulfoxide (26.5 mg/ml, 50 mM) was prepared freshly immediately before the labelling reaction. For labelling, 100 nMoles wheat germ agglutinin (representing approx. 1.5 μMoles amino functions) were reacted with 500 nMoles (3b) in a final volume of 400 μl 100 mM sodium tetraborate, pH 8.2, containing 2.5% (v/v) DMSO. The solution was incubated on an end-to-end mixer for 90 min at RT and the labelling reaction was terminated by addition of glycine (2.5 μMoles) in sodium tetraborate buffer, pH 8.2, resulting in a final volume of 1 ml. Mixing was continued overnight at 4° C. before the solution was dialyzed (MWCO 3,500) against sodium tetraborate buffer. The concentration of the labelled WGA (2,4-D-WGA) was determined by standard assays, the 2,4-D-WGA was snap-frozen in liquid nitrogen and stored at −80° C. 2,4-D-labelled WGA was utilized in a histological experiment to visualize Golgi membranes. Human colon carcinoma cells (cell line Caco-2, clone C2BBel; ATCC, Manassas, Va., USA) were grown for 48 or 72 h on sterile glass coverslips (Bellco Bio-technology, Vineland, N.J., USA) and subsequently treated at room temperature as follows. Cells were rinsed 3× with Dulbecco's phosphate buffered saline (D-PBS: 2.7 mM KCl, 1.5 mM KH₂PO₄, 136 mM NaCl, 8.1 mM Na₂HPO₄), fixed for 10 min with 4% (w/v) paraformaldehyde in D-PBS, and then permeabilized by incubating them for 5 min in 4% paraformaldehyde in D-PBS containing 0.1% (v/v) Triton X-100. Cells were rinsed with D-PBS, incubated for 10 min with 50 mM glycine in D-PBS and rinsed again with D-PBS. They were blocked twice with 10% (v/v) fetal bovine serum (FBS) in D-PBS each for 30 min. Subsequently, cells were incubated for 1 h with 15 μM (0.5 mg/ml) 2,4-D-WGA in 5% (v/v) FBS in D-PBS (FBS/PBS) in a humidified chamber to visualize plasma and Golgi membranes, before they were washed 3× for 15 min with D-PBS under mild rocking. To detect the 2,4-D label the cells were incubated for 1 h with 2,4-D-specific monoclonal antibody (clone E2/G2; 3.4 μg/ml in FBS/PBS) in a humidified chamber. Again, the cells were washed 3× for 15 min with D-PBS under mild rocking followed by incubation with AlexaFluor546-labelled goat anti mouse antibody (4 μg/ml in FBS/PBS; Invitrogen, Carlsbad, Calif., USA) for 1 h in a humidified chamber. The cells were washed 3× for 15 min with D-PBS under mild rocking. For nuclear staining the cells were incubated for 5 min with 4′,6-diamidino-2-phenylindole dihydrochloride (DAPI) (300 nM in D-PBS). The coverslips were rinsed 2× each with D-PBS and water and finally mounted onto glass examination slides with Mowiol containing 2.5% (w/v) 1,4-diazabicyclo[2.2.2]octane (DABCO). The coverslips were examined using a Nikon Diaphot300 microscope (Nikon, Düsseldorf, FRG) equipped with a Polaroid DMC2 camera (Polaroid, Dreieich-Sprendlingen, FRG) (FIG. 5).

### Example 19

### Labelling of DNA with 2,4-D-dUTP Derivatives (5a, 5b, 5c)

To label a DNA-fragment a murine prion protein gene fragment was amplified by polymerase chain reaction (PCR). As template DNA the vector pET15b (Merck Biosciences, Nottingham, UK) containing the cDNA coding for murine prion protein amino acids 90-231 (Schwarz A, Kralke O, Burwinkel M, Riemer C, Schultz J, Henklein P, Bamme T, Baier M (2003). Immunisation with a synthetic prion protein-derived peptide prolongs survival times of mice orally exposed to the scrapie agent. Neurosci Lett. 350:187-9.) was used. The primers for the PCR amplification were specific for the T7-promotor and T7-terminator (forward primer: 5′-TAATACGACTCACTATAGGG-3′; reverse primer: 5′-TGGCAGCAGCCAACTCAGC-3′; IBA, Göttingen, FRG). 1 μl template DNA solution (20 ng DNA) was added to the PCR mixture, containing 25 μMoles of each primer, 1×Taq DNA Polymerase buffer (New England Biolabs, Ipswich, Mass., USA), 3.5 U Taq DNA Polymerase (New England Biolabs), and a deoxynucleotide mix of dATP, dGTP and dCTP (each 10 nMoles) and dTTP (9.6, 8 or 6 nMoles) (Sigma-Aldrich, Taufkirchen, FRG). 2,4-D-labelled dUTP-derivatives (5a, 5b or 5c) were used to label the PCR-fragment. 1, 5 or 10 μl of the product fractions (approximately 0.4, 2 or 4 nMoles), which had eluted from the anion exchange column at about 57% (5a), 48% (5b) or 85% (5c) of eluent B (1 M NH₄HCO₃; see general description 5), and had been dissolved in 1 ml water after lyophilization, were added to the PCR mixture. Deionized water was added to obtain a total volume of 50 μl. As positive control analogous mixtures were prepared with various dTTP:dUTP ratios (10:0, 9.6:0.4, 8:2, or 6:4 nMoles), using non-labelled dUTP (Sigma-Aldrich). The PCRs were carried out in a Primus 25 Cycler (Peqlab Biotechnologie, Erlangen, FRG) using the following program: initial heating at 95° C. for 2 min, followed by 35 cycles of denaturation at 95° C. for 1 min, primer annealing at 51.5° C. for 1 min, and primer extension at 75° C. for 1 min, with an additional 5 min at 75° C. following the last cycle. The amplicon size of about 630 base pairs was verified by electrophoresis in a 1.5% (w/v) agarose gel and ethidiumbromide staining (marker: 2-log DNA ladder (New England Biolabs)).

Incorporation of 2,4-D-labelled dUTP-derivatives (5a, 5b or 5c) was verified in Southern Blot analyses. The PCR products were purified with a PCR Purification Kit according to the manufacturer's instructions (Qiagen, Hilden, FRG). 35 ng of a PCR-fragment which were either unlabelled or had been labelled with (5a), (5b) or (5c), were separated in a 1.5% (w/v) agarose gel and stained with ethidiumbromide. The fragments were transferred to Nytran SuPerCharge nylon membranes with a Turboblotter system according to the manufacturer's instructions (Whatman, Schleicher & Schuell, Dassel, FRG). The membranes were blocked in tight fitting trays for 2 h at RT in 1% (w/v) casein (Hammarsten grade, BDH, Poole, UK) in Dulbecco's phospate-buffered saline, pH 7.4 (D-PBS: 2.7 mM KCl, 1.5 mM KH₂PO₄, 136 mM NaCl, 8.1 mM Na₂HPO₄) (casein/PBS) and subsequently incubated for 1 h at RT in 2 μg/ml anti-2,4-D antibody (clone E4/C2) in casein/PBS. The membranes were washed 5× for 5 min with D-PBS containing 0.05% (v/v) Tween 20 and subsequently incubated for 45 min at RT in 0.2 μg/ml AlexaFluor680-labelled anti-mouse IgG antibody (Invitrogen, Carlsbad, Calif., USA) in casein/PBS. After another washing cycle (5×5 min with D-PBS) fluorescence was read out on a fluorescence imager (Odyssey Infrared Imager, LI-COR Biosciences, Lincoln, Nebr., USA) and quantified with an appropriate image analysis software (Odyssey Software v.1.2) (FIG. 6).

The detection limits of the 2,4-D-labelled DNA-fragments were determined in dot blot analyses. DNA-fragments, which had been amplified in the presence of 10 μl (5a) or 5 μl (5b) were purified as described and their concentrations were determined in standard assays. The fragments were serially diluted and equal amounts of the diluted solutions (10,000-0.61 pg DNA/dot) were immobilized onto Nytran SuPerCharge nylon membranes (Whatman, Schleicher & Schuell). The membranes were blocked in tight fitting trays for 2 h at RT in casein/PBS and subsequently incubated for 1 h at RT in 2 μg/ml anti-2,4-D antibody (clone E4/C2) in casein/PBS. The membranes were washed 6× for 10 min with D-PBS containing 0.05% (v/v) Tween 20 and subsequently incubated for 60 min at RT in 0.2 μg/ml AlexaFluor680-labelled anti-mouse IgG antibody (Invitrogen) in casein/PBS. After another washing cycle (6×10 min with D-PBS) fluorescence was read out on a fluorescence imager (Odyssey Infrared Imager, LI-COR Biosciences) and quantified with an appropriate image analysis software (Odyssey Software v.1.2) (Table 9).

TABLE 9
Detection limits of DNA-fragments labelled with
2,4-dichlorophenoxyacetic acid-(2,4-D)-deoxyuridine
triphosphate (dUTP) derivatives

| Incorporated nucleo- | Detection limit^{(a)} | Detection limit |
|---|---|---|
| tide-derivative | [pg DNA] | [aMole DNA] |
| 2,4-D-dUTP (5a) | 97.6 ± 19.5 | 235.2 ± 47.1 |
| 2,4-D-C6^{(b)}-dUTP (5b) | 12.5 ± 4.4^{(c)} | 30.1 ± 10.6^{(c)} |

^{(a)}The murine prion protein gene fragment 90-231 (629 bp) was amplified in the presence of the 2,4-D-labelled deoxyuridine triphosphate (dUTP) derivatives (5a) or (5b) in a polymerase chain reaction (PCR). The amplified DNA-fragments were purified, serially diluted and immobilized on nylon membranes as described. The DNA-fragments were detected with anti-2,4-D primary antibody (clone E4/C2) and AlexaFluor680-labelled secondary antibody. The lower detection limits (LDL) (arithmetic mean ± SEM) above cut-off are given in pg labelled DNA and converted into attomole labelled DNA: LDL (aMole DNA) = [LDL (g DNA)/(660 (g/Mole bp) * 629 (bp))] * 10¹⁸. The cut-off was calculated according to the procedure described by Frey et al. (Frey A, Di Canzio J, Zurakowski D (1998). A statistically defined endpoint titer determination method for immunoassays. J Immunol Methods. 221: 35-41.).
^{(b)}Aminohexanoyl-(C6)-Spacer;
^{(c)}detection limits for 2,4-dichlorophenoxyacetic acid labels that are significantly lower than those after labelling with (5a) (unpaired two-tailed t-test, P = 0.0054);
bp: base pairs.

### Example 20

### Labelling Efficacy of Polypeptidic Substrate Molecules with (3b)

A stock solution of (3b) (20-50 mM) was prepared freshly for each labelling experiment in anhydrous dimethylsulfoxide (DMSO). Stock solutions of proteinaceous substrate molecules, namely insulin (200 μg/ml, 35 μM; bovine insulin, MW 5733.49; Sigma-Aldrich, Taufkirchen, FRG), ubiquitin (1 mg/ml, 120 μM; bovine ubiquitin, MW 8565; Sigma-Aldrich) and Phl p 2 (1.3 mg/ml, 110 μM; recombinant His6-tagged grass pollen allergen 2 from Phleum pratense, MW 12214; Suck R, Petersen A, Weber B, Becker W M, Fiebig H, Cromwell O (2004). Analytical and preparative native polyacrylamide gel electrophoresis: Investigation of the recombinant and natural major grass pollen allergen Phl p 2. Electrophoresis. 25:14-9.), were prepared in 50 mM phosphate buffer, pH 7.2. For labelling, insulin (5 nMoles, representing 15 nMoles amino functions), ubiquitin (5 nMoles, representing 40 nMoles amino functions), or Phl p 2 (5 nMoles, representing 45 nMoles amino functions), were reacted with various molar excesses of (3b) in a final volume of 50-200 μl 50 mM phosphate buffer, pH 7.2, containing no more than 2% (v/v) DMSO in any experiment. For insulin labelling 0.02 mM ethylenediaminetetraacetic acid (EDTA) was added to all buffers. The solutions were mixed on an end-to-end mixer for 90 min at RT and the labelling reactions were terminated by addition of glycine (1 mMole) in phosphate buffer, pH 7.2. Mixing was continued for 60 min at RT before the solutions were dialyzed against phosphate buffer, pH 7.2. After another dialysis against water the heterogeneity of the samples and the molecular masses of the products were determined in MALDI-TOF-MS analyses applying a Bruker-Reflex II instrument (Bruker-Daltonik, Bremen, FRG) and dihydroxybenzoic acid as matrix. The mean labelling degree was calculated as described elsewhere (Olivier V, Meisen I, Meckelein B, Hirst T R, Peter-Katalinic J, Schmidt M A, Frey A (2003). Influence of targeting ligand flexibility on receptor binding of particulate drug delivery systems. Bioconjug Chem. 14:1203-8.) (FIG. 7).

1. Kit comprising,
at least one labelling compound characterized by formula (I: 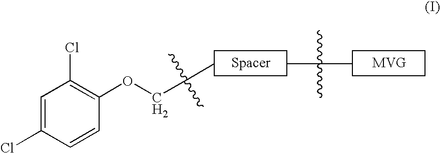
wherein the labelling compound is stable in water and soluble, and wherein the “Spacer” comprises 1 to 25 identical or different protected or unprotected amino acids, nucleotides, saccharides, polyoles or residues selected from the following group: 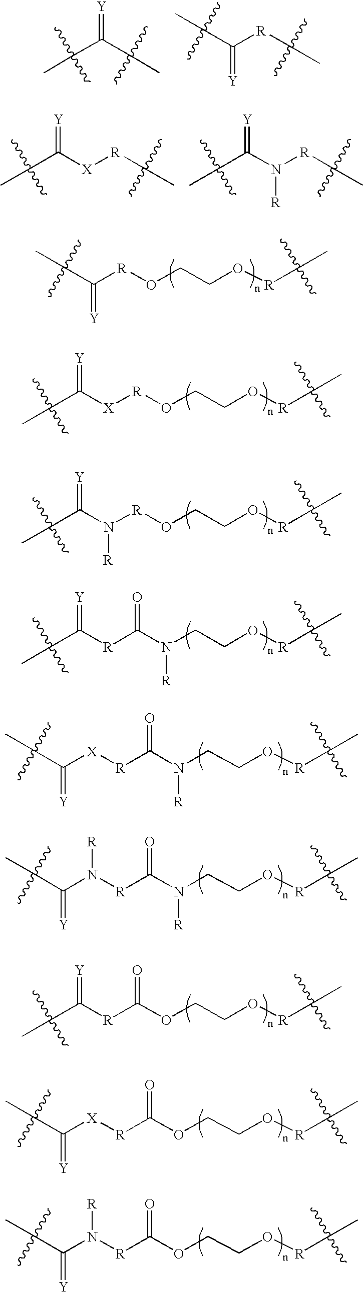 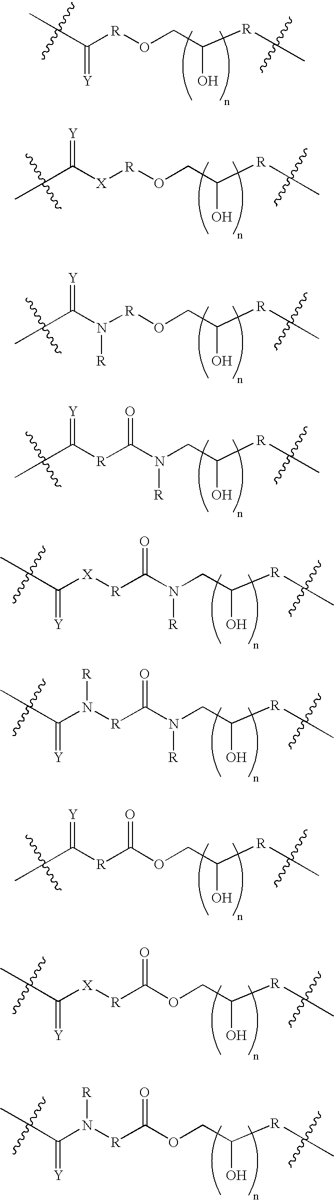
wherein X and Y, independently from each other, can be —O— or —S—, and n is an integer in the range of 1-15,
wherein the “label mediating group” (MVG) is selected from the following group: 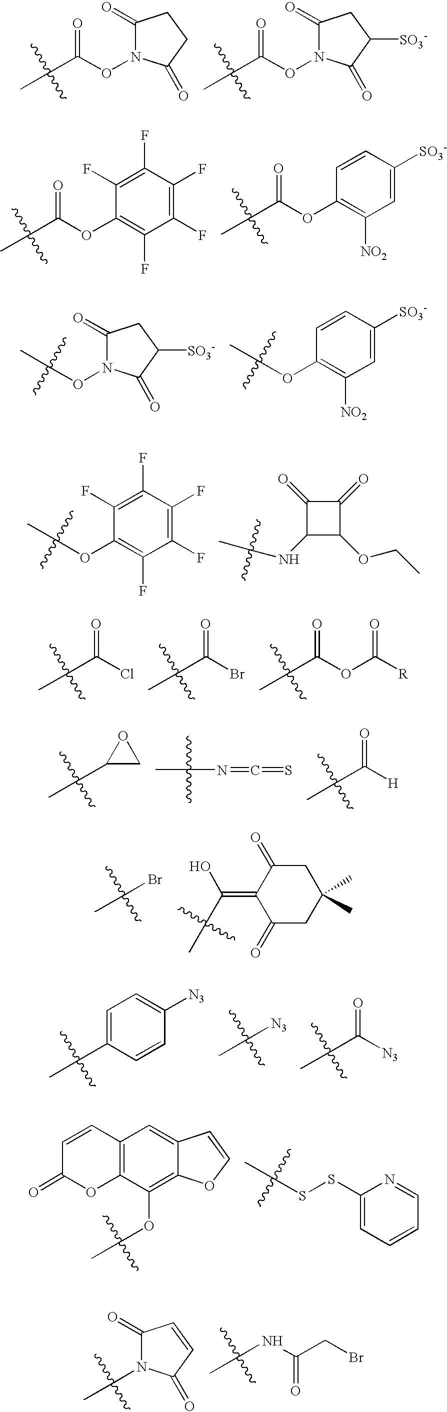 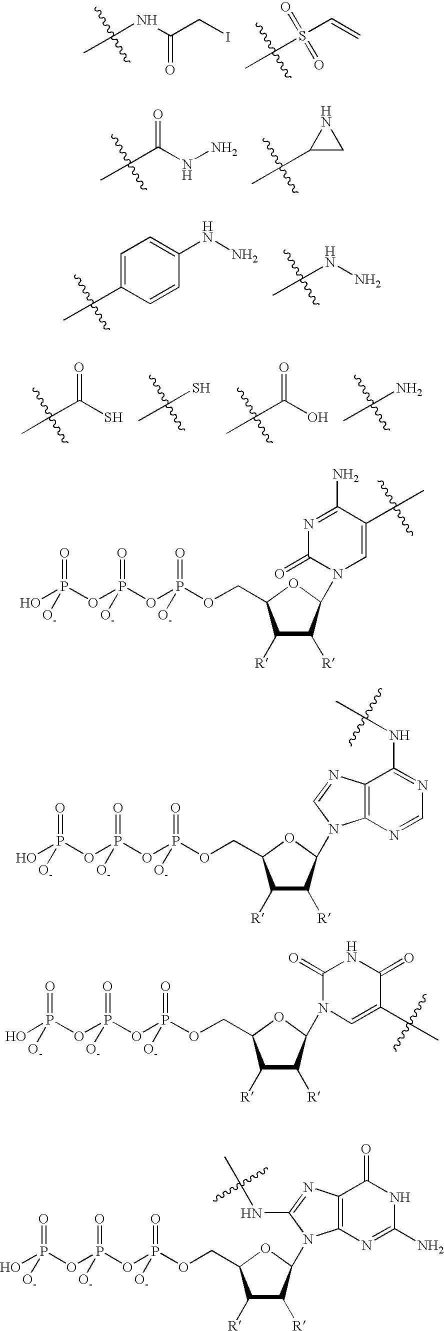 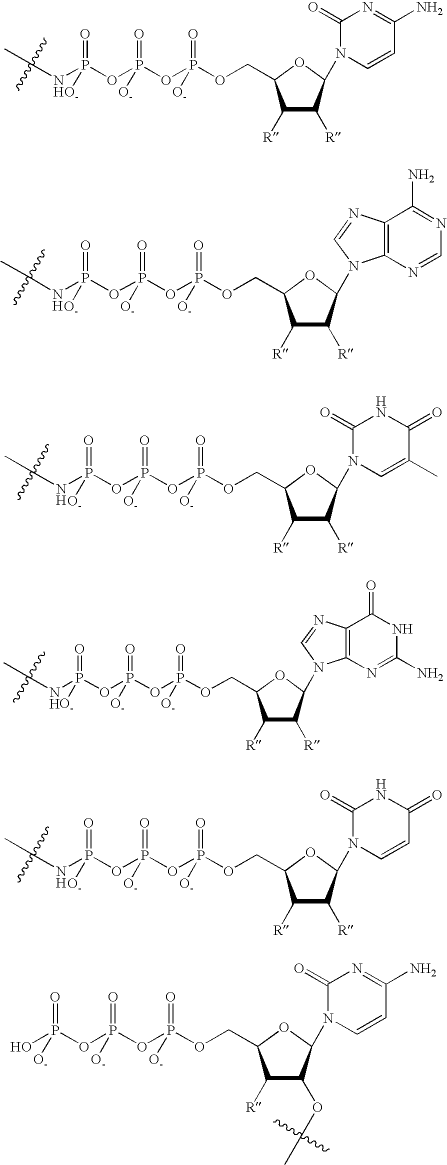 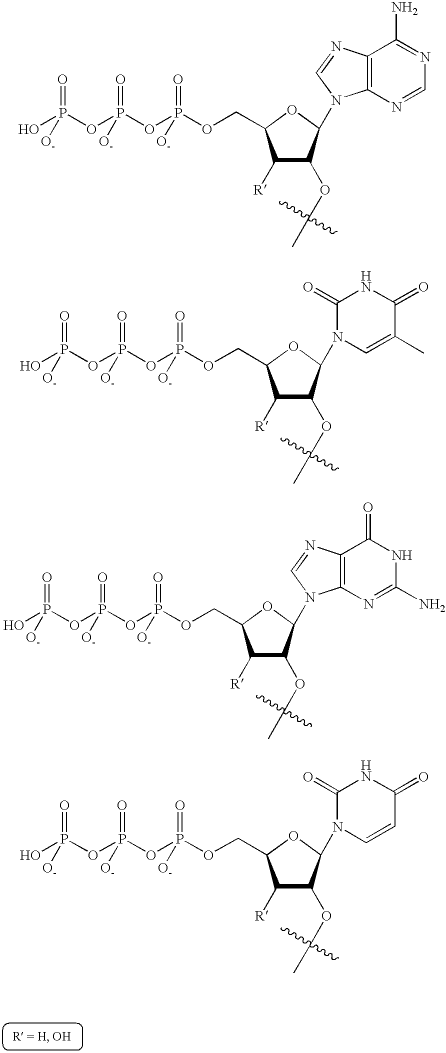 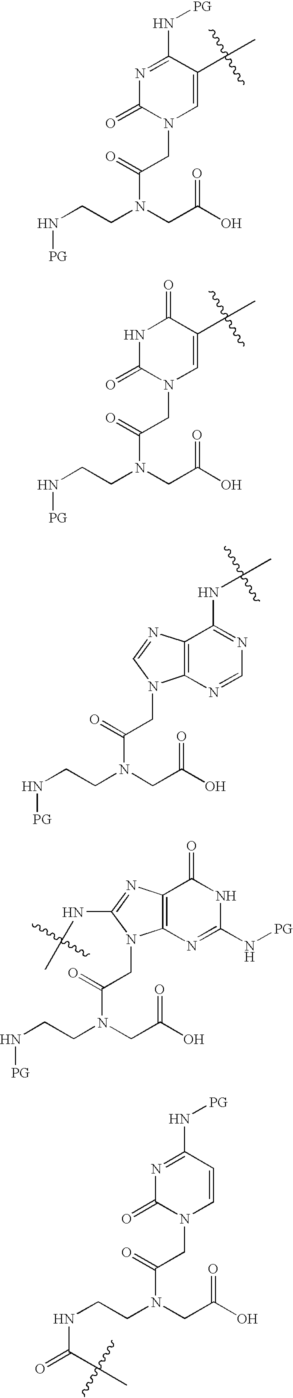 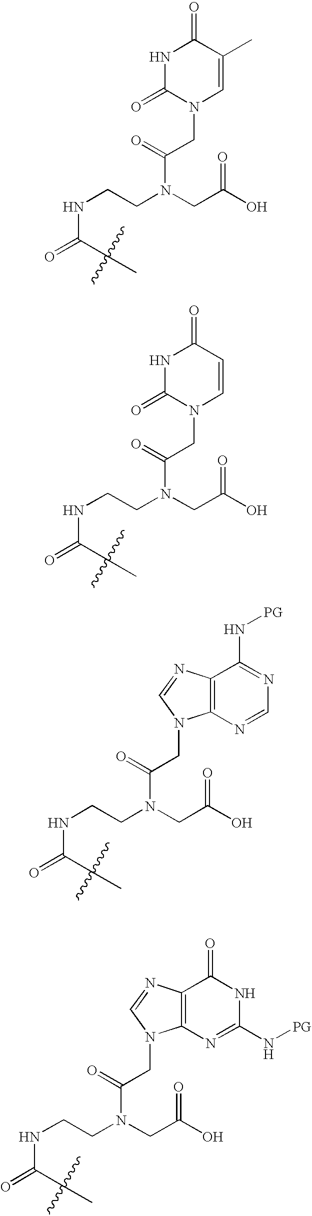 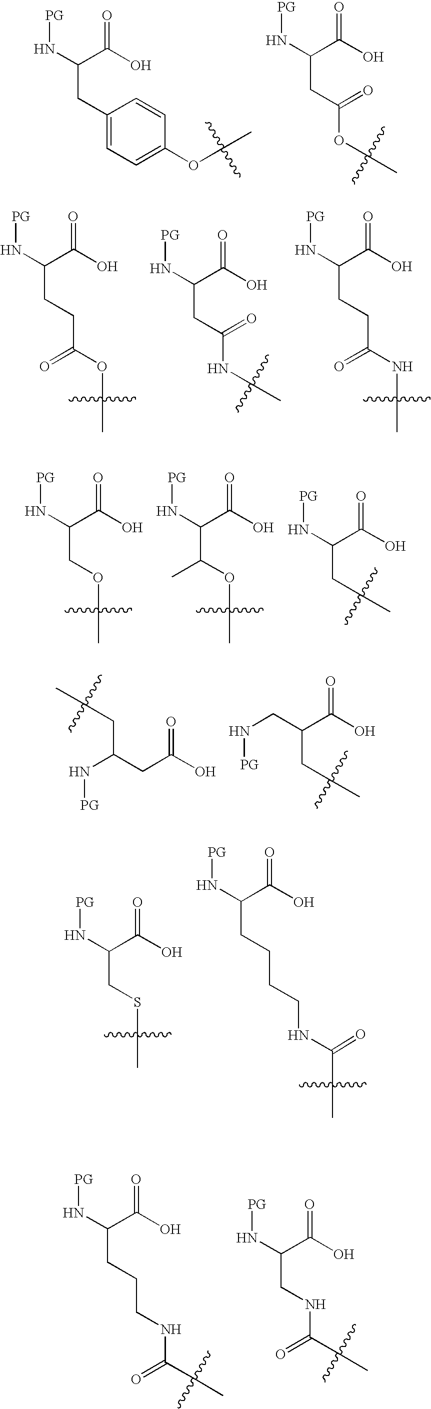 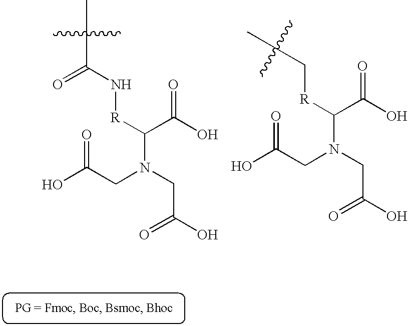
wherein R are independently from each other identical or different residues selected from the following group:
—H, linear, branched or cyclic alkyl residue or alkoxy residue comprising 1 to 15 carbon atoms, linear or branched alkenyl residue comprising 2 to 15 carbon atoms, protected or unprotected amine,
further comprising polyclonal antibodies, monoclonal antibodies, or fragments thereof, e.g. monovalent Fab or divalent (Fab)₂ fragments, that are suitable to bind to the labelling compound.

2. Kit according to claim 1, wherein the labelling compound is selected from the group consisting of (n=1, 2, 3, . . . , 13):

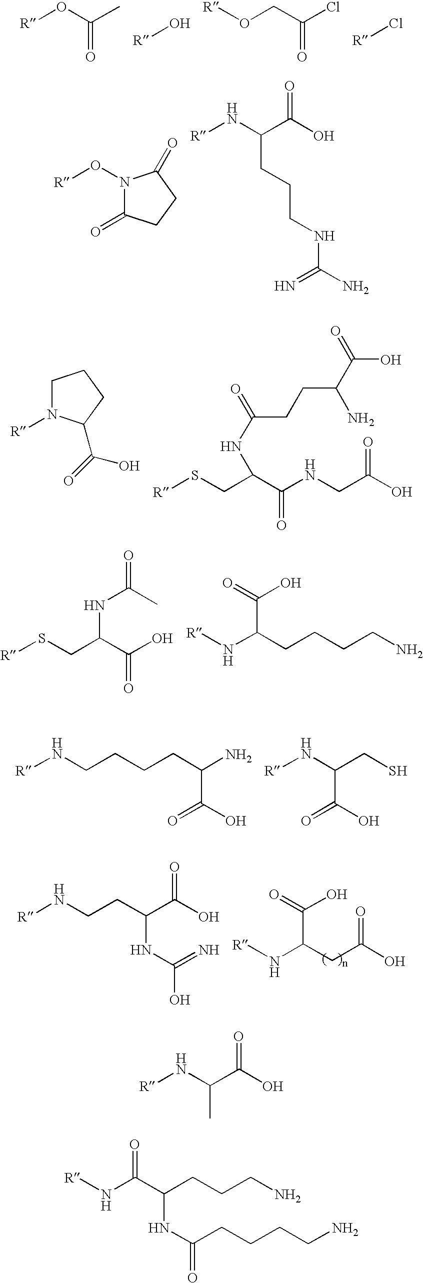

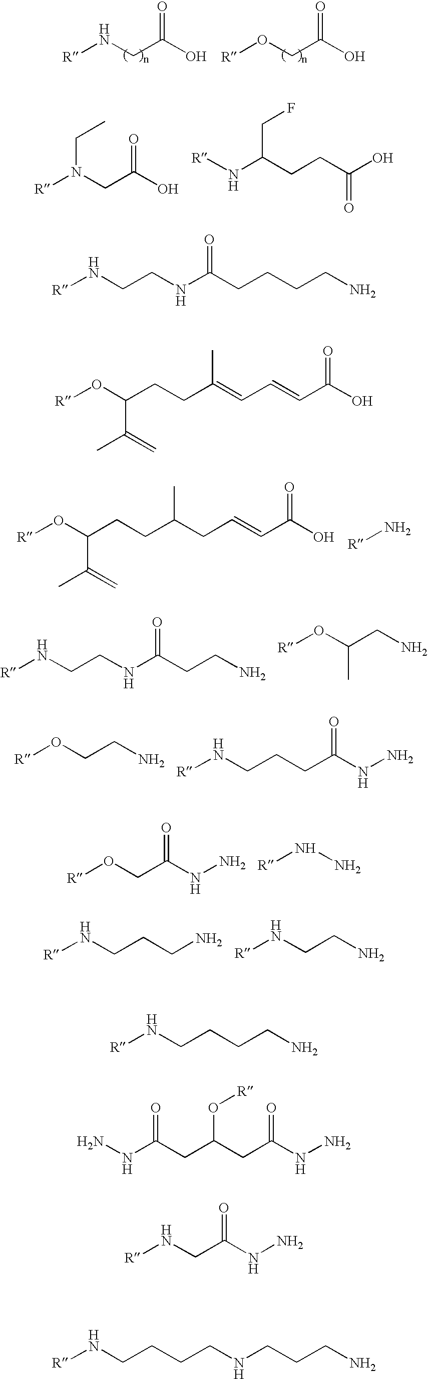

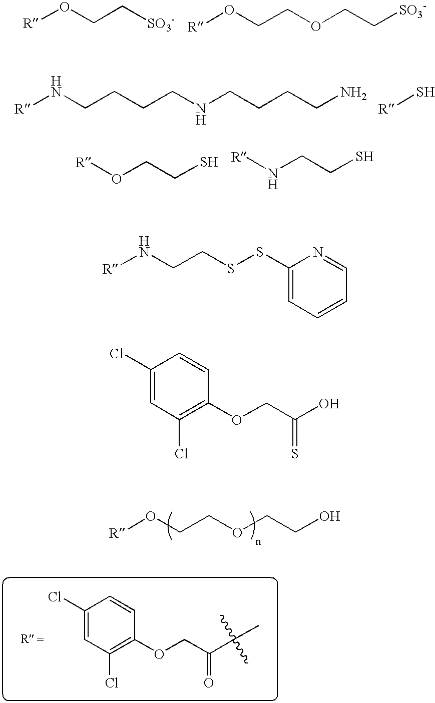

3. Kit according to claim 1, wherein the labelling compound is characterized by formula (II): 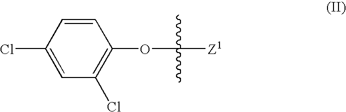
wherein Z1 is selected from the following substituents: 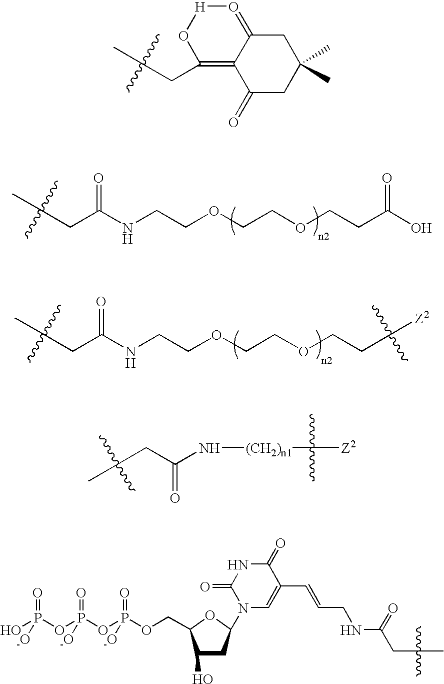
where n1 is 1-15,
wherein Z2 is selected from the following substituents 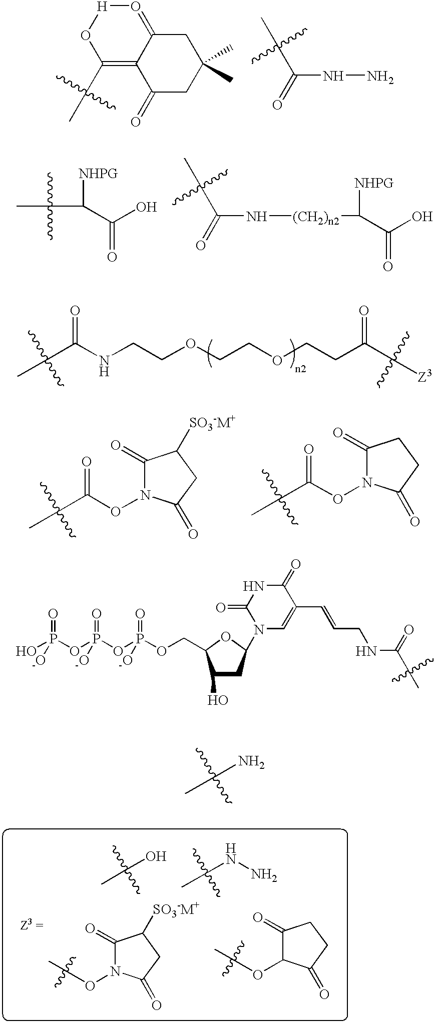
where n2 is 1-15,
wherein M⁺ is a monovalent, inorganic or organic cation,
wherein PG is an amino-protecting group.

4. Kit according to claim 1, wherein the labelling compound is characterized by formula (III) or (IV): 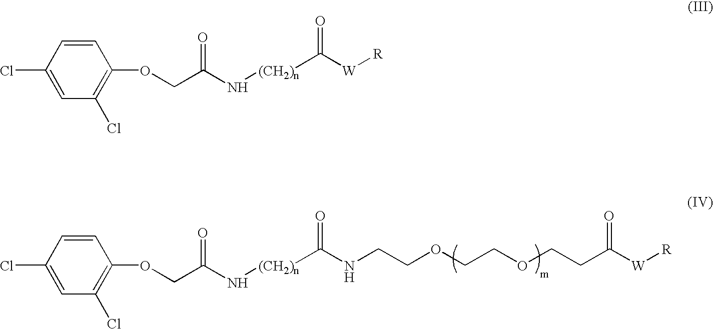
where n is 1, 2, 3, 4, 6, 7, 8, 9, 10, 11, 12, 13, 14, or 15, and
where m is 1, 2, 3, 4, 6, 7, 8, 9, 10, 11, 12, 13, 14, or 15,
wherein W is —O— or —NH— and R is hydrogen (—H), succinimidyl, sulfosuccinimidyl, amino (—NH₂), 5-allyl-2′-deoxyuridine-5′-triphosphatidyl or Fmoc-lysinyl.

5. Kit according to claim 3, wherein M⁺ is a lithium, potassium, ammonium, rubidium, caesium, sodium or tetraalkylammonium ion.

6. Kit according to claim 3, wherein the amino-protecting group is S-acetamidomethyl-(Acm), t-butyloxycarbonyl-(Boc), t-butyl- (tBu), trityl- (Trt), 2,2,4,6,7-pentamethyldihydrobenzofurane-5-sulfonyl- (Pbf), tosyl-(Ts), fluorenylmethoxycarbonyl-(Fmoc), (1,1,-dioxobenzo[b]thiophene-2-yl-methyl) oxycarbonyl- (Bsmoc), benzhydryloxycarbonyl- (Bhoc), or beta-2-adamantyl-(Ada).

7. Kit according to claim 3, wherein n1 is 5 or 10 and/or n2 is 4 or 11.

8. Kit according to claim 1, wherein the antibodies are antibodies produced by a hybridoma obtainable according to the method of Franek et al. (1994) J Agric Food Chem. 42:1369-1374.

9. Kit according to claim 1, further comprising buffers and/or solutions and/or reagents suitable to couple labeling compounds to substrate molecules and to perform detection assays with antibodies.

10. Use of a kit according to claim 1 for labelling and detecting substrate molecules.

11. Use of claim 10, wherein the substrate molecule is a macromolecule.

12. Use of claim 10, wherein the substrate molecule is a bio-genous macromolecule.

13. Use of claim 10, wherein the substrate molecule is an amino acid, a branched or unbranched oligopeptide, a polypeptide, a protein, a nucleic base, a nucleoside, a nucleotide, an oligonucleotide, a polynucleotide, a nucleic acid, a monosaccharide, an oligosaccharide, a polysaccharide, a lipid or a glycoprotein.

14. Use of claim 13, wherein the substrate molecule is a polypeptide comprising 2 to 50 amino acids.

15. Use of claim 13, wherein the protein has a mass of more than 5 kDa.

16. Use of claim 13, wherein the polynucleotide encompasses 2 to 100 nucleotides.

17. Use of claim 13, wherein the nucleic acid encompasses 100 to 5000 nucleotides.

18. Use of claim 13, wherein the oligosaccharide comprises 2 to 30 monosaccharides.

19. Use of claim 13, wherein the polysaccharide has a molecular mass of more than 5 kDa.

20. Use of a kit according to claim 1, for immunological assays, solid-phase supported diagnostic applications, enzyme-linked immunosorbent assays, hybridization assays, polymerase chain reactions and biological labelling and biological uptake experiments.
